SEQUENCE LISTING (4 sequences)

SEQ ID NO: 1 - DNA, 30 bp, Artificial Sequence
  Synthetic
tacgcgtgctagcgagctcattaatgcgat

SEQ ID NO: 2 - DNA, 32 bp, Artificial Sequence
  Synthetic
cgcattaatgagctcgctagcacgcgtatgca

SEQ ID NO: 3 - DNA, 29 bp, Artificial Sequence
  Synthetic
ggtcgactatcgtttccacgatcagcgat

SEQ ID NO: 4 - DNA, 29 bp, Artificial Sequence
  Synthetic
caagcttggatcactgtattcggctgcaa

(19) United States
(12) Patent Application Publication
(10) Pub. No.: US 2006/0003454 A1
(43) Pub. Date: Jan. 5, 2006

(54) Non-dividing donor cells for gene transfer

(75) Inventors: Hideki Suzuki, Madison, WI (US); Marcin Filutowicz, Madison, WI (US); Larry Anthony, Madison, WI (US)

Correspondence Address: MEDLEN & CARROLL, LLP, 101 Howard Street, Suite 350, San Francisco, CA, 94105, US

(73) Assignee: ConjuGon, Inc., Madison, WI (US)

(21) Appl. No.: 10/884,257

(22) Filed: Jul. 2, 2004

(51) Int. Cl. C12N 1/21; C12N 15/74

(52) U.S. Cl. 435/471; 435/252.3

(57) ABSTRACT

The present invention relates to compositions comprising conjugation-competent non-dividing cells for delivery of genes to bacterial cells by conjugation. The present invention also relates to methods of making, using, and storing such conjugation competent non-dividing cells.

### FIELD OF THE INVENTION

The present invention relates to compositions and methods for delivering genetic material to bacterial cells by conjugation with non-dividing donor cells.

### BACKGROUND OF THE INVENTION

Use of bacteria for numerous treatment purposes is well known in the art. For example, preparations of Lactobacillus acidophilus for use in human therapies is known (see, e.g., U.S. Pat. No. 5,032,399, Issued July, 1991 to Gorbach et al., and U.S. Pat. No. 5,733,568, issued March, 1998 to Ford). In addition, pharmaceutical preparations of Lactobacillus acidophilus are known (see., e.g., U.S. Pat. No. 4,314,995, issued Feb. 9, 1982 to Hata et al., “Pharmaceutical lactobacillus preparations”). Additional applications of bacteria in human therapeutics are described in U.S. Pat. No. 5,607,672 (Using recombinant Streptococcus mutans in the mouth to prevent tooth decay); U.S. Pat. No. 6,447,784 (Genetically modified tumor-targeted bacteria (Salmonella) with reduced virulence); U.S. Pat. No. 6,723,323 (Vibrio cholerae vaccine candidates and method of their constructing); U.S. Pat. No. 6,682,729 (A method for introducing and expressing genes in animal cells is disclosed comprising infecting the animal cells with live invasive bacteria); and U.S. Pat. No. 4,888,170 (relating to a vaccine for the immunization of a vertebrate, comprising: an avirulent derivative of a pathogenic microbe).

Uses of bacteria for a variety of other purposes, such as treatment of animal feed, are also known in the art (see, e.g., U.S. Pat. No. 5,549,890, issued Aug. 27, 1996 to Kubo, describing the use of Bacillis subtilis in livestock feed to produce a fattening feed; U.S. Pat. No. 4,138,498, issued Feb. 6, 1979 to Das, et al., describing the use of cultures of Megasphaera elsdenii and Selenomonas ruminantium as feed additives for ruminant animals to improve dietary adaptation.

In some particularly useful treatments, bacteria are used to deliver genetic material to targeted recipient bacteria via conjugation. See, e.g., PCT Publication WO 02/18605, incorporated herein by reference in its entirety. In such treatments, the bacteria can be used to deliver genes that can alter the function of the targeted recipient bacteria, or that are lethal to the recipient bacteria.

For some applications, it is desirable to use whole, viable bacterial cells. Live bacteria can be designed to colonize a targeted site, e.g., to compete out the residing harmful bacteria or to destroy tissue harmful to a host body (e.g., tumors). However, for many applications, it is desirable to use cells that cannot grow in the environment into which they are to be introduced. For applications where growth of the delivering bacteria is not desirable, a containment system is generally used. Such containment systems have generally been of two types: “passive containment” and “active-containment”. Passive containment systems generally relate to the use of microbes termed “conditional mutants”, such that the delivering microbe can only survive in particular conditions. One example of such a conditional mutant is an auxotroph, which is a nutrition-requiring microbe that cannot biosynthesize an essential component for its survival. Such auxotrophs can only survive if the component is supplied from the environment (Sorensen et al., Appl. Environ. Microbiol. 66:1253-1258 [2000]; Bron et al., Appl. Environ. Microbiol. 68:5663-5670 [2002]). Numerous auxotrophs are known in the art and can be obtained easily for use in a passive-containment system.

Although useful in properly selected or controlled environments, nutrition based-passive containment is less effective in environments where a broad array of nutrients is readily available. For example, in an animal tissue many auxotrophs can readily grow because all essential nutrients are available. In such a nutrient rich environment an active containment system may be preferred. Active-containment systems utilize conditional expression of toxic peptides to kill the organism to be contained. (Schweder et al., Appl. Microbiol. Biotechnol. 42:718-723 [1995]; Torres et al., Environ. Microbiol. 2:555-563 [2000]). Such active-containment systems may be used to control delivering bacteria. However, the toxic peptides used for active-containment systems are generally derived from pathogenic organisms. The presence of such peptides may be undesirable for some applications of bacterial delivery, e.g., in therapeutic applications, thus limiting the utility of the active containment systems for these applications.

Another approach to containment involves use of cells that have been modified to remove their genetic material, so that cell division and growth is impossible. Such nonviable cells include bacterial ghosts, minicells and maxicells.

Bacterial ghosts are the empty cell envelopes of Gram-negative bacteria. Bacterial ghosts are useful as carriers and delivery vehicles of a variety of compounds, drugs, and the like. However, because they lack essentially all cellular metabolic capabilities, bacterial ghosts are generally unable to transfer material by conjugation.

Minicells lack chromosomal DNA but retain other cellular metabolic capacities. Minicells are generated by special mutant cells that undergo cell division without DNA replication. Minicells neither divide nor grow, but minicells that possess transmissible plasmids are capable of conjugal replication and transfer of plasmid DNA to living recipient cells. (Adler et al., 1970, supra; Frazer and Curtiss, 1975, supra; U.S. Pat. No. 4,968,619, supra). However, preparation of minicells is time consuming.

Maxicells are cells that are treated so as to destroy their chromosomal DNA, while retaining the function of plasmids that they contain. Maxicells can be obtained from a strain of E. coli that carries mutations in the key DNA repair pathways (e.g., mutations in recA, uvrA and phrB genes). Because maxicells lack so many DNA repair functions, the chromosomal DNA cannot replicate and the cells cannot divide after exposure to doses of ultraviolet (UV) light. Plasmid molecules in the treated cells are much smaller than the chromosomal DNA and are less likely to be damaged by the UV light. Plasmids that do not receive a UV hit will continue to replicate. Plasmid-directed transcription and translation can occur efficiently under such conditions (Sancar et al., J. Bacteriol. 137: 692-693, 1979), and the proteins made prior to irradiation can sustain some level of cellular mtabolism.

After UV irradiation, maxicells are further incubated to facilitate extensive degradation of the chromosomal DNA of the bacteria. This is an essential part of the process of making maxicells, to prevent de novo protein synthesis from the chromosome from interfering with the analysis of proteins arising from plasmid function. To extensively degrade the chromosomal DNA in the UV-irradiated bacterial cells, extended incubation of the cells is required (Heinemann and Ankenbauer, Mol Microbiol., 10:57-62 [1993]), which damages cellular metabolic capabilities. Significant reduction in conjugation efficiency of the maxicells is unavoidable after this extended incubation, possibly due to degradation of conjugation machinery synthesized prior to the irradiation. As indicated in Table 2 of Heinemann, conjugation efficiency drops dramatically (about three orders of magnitude) after maxicell preparation.

Thus, there is a need for non-dividing cells that retain a high level of metabolic function. In particular, there is a need for non-dividing cells that retain a high level of conjugation-competency. There is a further need for systems and methods to produce and store non-dividing cells with the minimal loss of the conjugation-competency.

### SUMMARY OF THE INVENTION

Compositions and methods for generating conjugation-competent non-dividing donor cells, and their prolonged storage are provided. In some embodiments, the non-dividing donor cells are used as a delivery system. In some preferred embodiments, the non-dividing donor cells find use as a system for delivering genetic material to a recipient cell. In particularly preferred embodiments, the recipient cell is a pathogenic bacterium.

The present invention provides methods to store the conjugation-competent non-dividing cells for extended time periods.

In some embodiments, the present invention comprises a composition comprising a non-dividing cell derived from a bacterial cell harboring a defective DNA repair system. In some embodiments, the bacterial cell comprises one or more mutations in the DNA repair system. The present invention is not limited to any particular mutation in the DNA repair system. In some embodiments, the composition comprises a bacterial cell harboring a recA mutation. In some embodiments, the composition comprises a bacterial cell comprising one or more other mutations in the DNA repair system Such mutations include but are not limited to phrB and uvrA (Sancar et al., J Biol Chem, 259:6033-6038 [1984]; Thomas et al., J. Biol Chem, 260:9875-9883 [1985]). The product of phrB is essential for photoreactivation by catalyzing reversion of a UV-damaged DNA (e.g., cyclobutane pyrimidine dimer). The product of uvrA repairs the UV-damaged DNA through different system (e.g., nucleotide excision repair). Eliminating one or both of the above gene functions would reduce survival of recA mutants.

In some embodiments, the non-dividing cell further comprises genes for conjugation, e.g., transfer or tra genes. In some embodiments, the tra genes are from plasmid RK2, however, the present invention is not limited to tra genes derived from RK2. For example, since a large number of conjugation systems are know to the art, any one or a combination of them may be applicable. The present invention is not also limited to the location of the tra genes, and they can be integrated on the genome and/or on non-chromosomal DNA.

In some embodiments, irradiation is used to damage DNA of bacteria to produce the non-dividing cells of the present invention, e.g., to disable further bacterial cell division without disabling other metabolic capacities, such as conjugation. In some embodiments, UV light irradiation is used. In other embodiments, another DNA-damaging irradiation, including but not limited to gamma ray irradiation, is used.

Methods to generate non-dividing cells are not limited to DNA-damaging irradiation. Conditional expression of toxin genes may be used to generate non-dividing conjugation-competent cells. These methods may be combined, or they may be used independently. The toxins, for example, may be bacterial colicins (Riley and Wertz, Annu Rev Microbiol. 56:117-137 [2002]; Lazdunski et al., J. Bacteriol., 180:4993-5002 [1998]).

Colicins D and E3 are RNase enzymes that prevent protein synthesis by cleavage of arginine tRNA or the bacterial ribosome, respectively (Tomita et al., Proc Natl Acad Sci 97:8278-8283 [2000]; Bowman et al., Proc Natl Acad Sci 68:964-968 [1971]). Colicin E7 is a non-specific DNAse enzyme which degrades chromosomal and plasmid DNA (Chak et al., Proc Natl Acad Sci 93:6437-6442 [1996]; Kuhlmann et al., J Mol Biol 301:1163-1178 [2000]). Colicins are extremely potent antibacterials and are lethal to bacterial cells at extremely low concentrations. Expression of colicins such as those described above would cause immediate cessation of cell growth and would prevent further cell division. However, these cells should retain basic metabolic capacities such as conjugation for a period of time.

In some embodiments, bacterial cells are irradiated with UV light in a Petri dish on a rotary shaker. The method of irradiation is not limited to this format. It is contemplated that any method of exposing cells to irradiation may be adapted to the methods of the present invention. For example, cells may be passed by a radiation source in a controlled manner. By way of example, and not intending to limit the invention to any particular manner of exposing cells to a radiation source, FIG. 6 diagrams one configuration whereby cells are passed through irradiation-permeable tubing for preparing larger amount in a controlled manner.

The present invention provides methods of producing non-dividing cells that retain cellular metabolic function. In some embodiments, the present invention provides methods of producing non-dividing cells that retain conjugation competencies. In some embodiments, the non-dividing cells are produced by providing a conjugation-competent bacterial cell that is deficient in one or more DNA repair systems such that the cell's ability to repair DNA damage is substantially impaired, exposing the bacterial cell to DNA damaging conditions whereby the chromosomal DNA of said bacterial cell is damaged sufficiently to prevent cell division, and treating the bacterial cell having damaged chromosomal DNA under conditions wherein cellular metabolic function is preserved. In preferred embodiments, the cells having damaged chromosomal DNA are treated under conditions wherein conjugation competency is preserved. In particularly preferred embodiments, the non-dividing cell produced by the treatments has conjugation-competency that is substantially similar to the conjugation competency of an untreated bacterial cell.

In some embodiments, the DNA damaging conditions comprise irradiation. In some preferred embodiments, the irradiation comprises irradiation by ultraviolet light. In other preferred embodiments, the irradiation comprises irradiation by gamma rays.

In some embodiments, the bacterial cells having damaged chromosomal DNA are chilled to preserve metabolic function and/or conjugation competence. In some embodiments, the cells are chilled. Chilling is not limited to any particular temperature or range of temperatures. In some embodiments the cells are chilled to a temperature of about 0° C. to 10° C. In preferred embodiments, the cells are chilled to about 0° C. to 5° C. In particularly preferred embodiments, the cells are chilled to a temperature of about 0° C. to 1° C.

In some embodiments, the bacterial cells having damaged chromosomal DNA are chilled to a temperature below 0° C. In some embodiments, the cells are frozen. In some preferred embodiments, the cells are frozen to very low temperatures below about −50° C.

In some embodiments the cells are chilled immediately after irradiation. In some embodiments, the cells are chilled before irradiation and the chilling is maintained after irradiation.

In some embodiments the bacterial cell used in the methods of the present invention is Gram-positive. In some embodiments, the bacterial cell is selected from the group consisting of Lactobacillis acidophilis, Lactococcus lactis, Lactobacillus plantarum, Bacillus subtilis, Staphylococcus species, Streptococcus species. In other embodiments, the bacterial cell used in the methods of the present invention is Gram-negative. In some embodiments the bacterial cell is selected from the group consisting of Escherichia coli, Helicobacter pylori, Pseudomonas aeruginosa, Haemophilus influenzae, somnus and ducreyi, Klebsiella pneumoniae

In some embodiments, the bacterial cell used in the methods of the present invention comprises tra genes encoding components for conjugation. In some embodiments the tra genes are located on the chromosomal DNA of the bacterial cell, whereas in some embodiments the tra genes are located on a plasmid. In some preferred embodiments the tra genes are located on a transmissible plasmid. In other preferred embodiments the tra genes are located on a helper plasmid. In still other embodiments, one or more tra genes are located on both the bacterial chromosomal DNA and on one or more plasmids.

In some embodiments, the bacterial cell further comprises a transmissible element. In preferred embodiments, the transmissible element is DNA. In some particularly preferred embodiments, the said DNA is a plasmid.

In some embodiments the DNA transmissible element comprises an origin of transfer. In some preferred embodiments the origin of transfer is from a Gram-negative bacterium. In other preferred embodiments the origin of transfer is from a Gram-positive bacterium.

In some embodiments the present invention provides a method of producing a conjugation-competent non-dividing cell by providing conjugation-competent bacterial cells that are deficient in one or more DNA repair systems such that the cell's ability to repair DNA damage is substantially impaired, irradiating the conjugation-competent bacterial cells, such that the chromosomal DNA of the bacterial cell is damaged sufficiently to prevent cell division, wherein said irradiation comprises passing the bacterial cells past a radiation source to provide a controlled dosage of irradiation to the cells, and treating said bacterial cells having damaged chromosomal DNA under conditions wherein conjugation competency is preserved. In some preferred embodiments the dosage of irradiation received by said bacterial cells is controlled by the rate at which said bacterial cells and said radiation source pass each other. In particularly preferred embodiments the radiation source is stationary and said bacterial cells are moved past the radiation source, e.g., in a continuous flow in, for example, a tube that is transparent to the radiation.

The present invention provides composition comprising a non-dividing cell, wherein said the non-dividing cell is a bacterial cell deficient in one or more DNA repair systems, wherein the bacterial cell has been exposed to DNA damaging conditions wherein the chromosomal DNA of said bacterial cell is damaged sufficiently to prevent cell division, and wherein said bacterial cell has further been treated under conditions wherein cellular metabolic function is preserved. In preferred embodiments, the bacterial cell having damaged chromosomal DNA is treated under conditions wherein conjugation competency is preserved.

In some embodiments the non-dividing cells of the present invention further comprise one or more transfer genes conferring upon the cell the ability to conjugatively transfer a transmissible plasmid to at least one recipient bacterial cell, and further comprise at least one transmissible plasmid, wherein said transmissible plasmid comprises an origin of transfer (oriT) from which conjugative transfer of the transmissible plasmid initiates from the non-dividing cell to at least one recipient cell. In some embodiments, the oriT is from plasmid RK2

In some embodiments, the prepared non-dividing cells are stored. In some embodiments the non-dividing cells are stored with a preservative. For example, in some preferred embodiments, the non-dividing cells are stored in a glycerol-containing solution. In some embodiments, the preservative is a cryopreservative. In other embodiments, different cryopreservatives may be used, alone or in different combinations. A number of cryopreservatives are known in the art for cryopreservation of cells (e.g., glycerol, trehalose, sucrose, dimethylformoxide (DMSO) and ethylene glycol). (Hubalek, Cryobiol., 46:205-229 [2003]). In some embodiments, non-dividing cells are frozen using an ethanol-dry ice bath. In other embodiments, non-dividing cells are frozen in liquid nitrogen. Many methods of freezing cells are known in the art and the present invention is not limited to any particular method.

In some embodiments, the prepared non-dividing cells are stored frozen (e.g., below 0° C.). The present invention is not limited to any particular temperature below 0° C. for storage in a frozen state. In some embodiments the non-dividing cells are stored below about −20° C. In preferred embodiments the non-dividing cells are stored at a temperature below about −50° C. In particularly preferred embodiments the non-dividing cells are stored at a temperature below about −80° C.

The present invention is not limited freezing as a method of storage. Other methods of bacterial storage known in the art are applicable to the storage of the non-dividing cells of the present invention. For example, a freeze-drying (or lyophilization) process can be used to prepare bacteria for storage. Freeze-drying involves the removal of water from frozen bacterial cells by a process called sublimation. Freeze-dried bacterial suspensions can be stored without refrigeration and can be stored indefinitely at ambient temperatures. The process of freeze-drying (lyophilization) is a common technique for long-term storage of bacteria and has been fully documented in the scientific literature (Heckly, Dev Indust Microbiol., 26:379-395 [1985]; Miyamoto-Shinohara et al., Cryobiol., 41(3)251-255 [2000]; Nicholson, Dev Biol Standardization, 36:69-75 [1977]), each incorporated by reference herein. Dried suspensions of bacteria have been formulated into tablets, capsules, or bulk powders, for ease of use in applications.

### DESCRIPTION OF THE DRAWINGS

FIG. 1A is a schematic diagram depicting bacterial conjugation. FIG. 1B shows an example of exconjugants growing on a plate containing a combination of antibiotics that is toxic to the parent donor and recipient cells, and provides an example of an assay used to determine conjugation efficiency.

FIG. 2 depicts a schematic diagram of a process of generating non-dividing bacterial cells. In this experiment, the non-dividing cell conjugates efficiently with a recipient bacterium. The conjugation machinery synthesized from the RK2-derived tra genes encoded on its own plasmid. The conjugation machinery synthesized prior to UV irradiation facilitates conjugation from the non-dividing cells. UV light-irradiation damages chromosomal DNA. If the damaged DNA is left un-repaired, completion of DNA replication fails, resulting in a cell lacking the intact chromosomal DNA that cannot grow and divide. In the figure, large rectangular-circles indicate independent bacterial cells. The zigzag lines and small circles indicate chromosomal and plasmid DNA, respectively. Small circles on the chromosomal and plasmid DNA indicate sites of DNA damage generated by UV light-irradiation. Upon UV light-irradiation bacterial cells harboring mutation(s) in the DNA repair system quickly lose their viability.

FIG. 3 depicts maps of exemplary conjugative self-transmissible plasmids, and a non-self-transmissible plasmid. RK2 and pCON4-45 are self-transmissible plasmids, and all the tra genes essential for conjugation are encoded on their own plasmids. pCON1-64D is a non-self-transmissible plasmid, and it can be mobilized only when the products of the tra genes are supplied in trans within the same bacterial cell. oriT (in this case derived from RK2) is essential for mobilization of pCON1-64D. The function of the genes encoded in these regions are not associated with conjugation. Rep, the region essential for replication of the plasmid from the oriV region; oriT, the region where the single-stranded DNA transfer occurs upon conjugation; primase, the region essential for synthesizing the complementary DNA strand after the single-stranded DNA is transferred into a recipient cell with conjugation; TetR, tetracycline-resistance determinant; AmpR, ampicillin-resistant determinant; KanR, kanamycin-resistant determinant; Control, the region encoding genes to control the expression of genes on the RK2 plasmid; trfA, genes encoding an essential protein to initiate replication from oriV. Note that these plasmids are significantly different in their sizes; therefore sizes of the genes in this figure are not in scale.

FIG. 4 depicts a schematic of conjugative transfer of a self-transmissible plasmid RK2 from conjugation-competent non-dividing cells generated from JM109. The result demonstrates that the self-transmissible plasmid RK2 is conjugatively transferred using the conjugation machinery generated from its own plasmid, but not from the genome of the host cell.

FIG. 5 depicts a schematic of conjugative transfer of a non-self-transmissible plasmid pCON4-64d from conjugation-competent non-dividing cells prepared from S17-1. The result shows that the non-self-transmissible plasmid pCON1-64D is conjugatively transferred using the conjugation machinery generated from the genome of the host cell.

FIG. 6 depicts a schematic of one exemplary system for generating large quantities of non-dividing cells. Non-dividing cells may be generated by controlled exposure of DNA-damaging-irradiation while the bacterial cells are being passed through from a reservoir vessel to a collection and storage vessel.

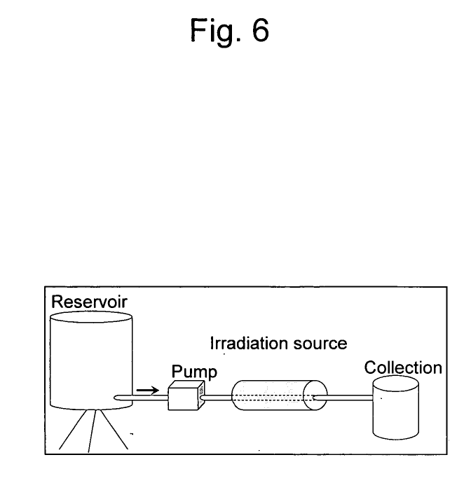

FIG. 7 shows a results demonstrating that the non-dividing cells could be stored in −80 C for extended periods without losing a significant level the conjugation-competency. Non-dividing cells were prepared from an E. coli strain S17-1 carrying a self-transmissible plasmid pCON4-45 (see FIG. 3). These non-dividing cells were frozen in ethanol-dry ice, and stored in −80 C, and their conjugation efficiencies were monitored after different storage times, using a filter conjugation method as described in Example 2. Conjugation efficiencies obtained from the stored non-dividing cells were standardized using freshly grown conjugation donor (S17-1 carrying pCON4-45) at each time point. Upon freezing, a slight reduction of conjugation efficiency was observed. However, no further change was observed in their conjugation efficiencies after extended storage periods tested.

FIG. 8 shows a result demonstrating that the non-dividing cells could be stored in a lyophilized form. Lyophilized non-dividing cells (lane 1) was used as conjugation donors, and compared to that of freshly cultured donor cells (lane 2). Non-dividing cells were prepared from an E. coli strain S17-1 carrying a self-transmissible plasmid pCON4-45 (see FIG. 3). These non-dividing cells were re-suspended in 0.9% NaCl, and quickly frozen in ethanol-dry ice. The frozen non-dividing cells were lyophilized, and kept at 4 C. After a storage period, the lyophilized cells were regenerated by adding water, and their conjugation efficiencies were monitored after different storage times, using a filter conjugation method. Conjugation efficiencies obtained from the stored non-dividing cells were standardized using freshly grown conjugation donor (S17-1 carrying pCON4-45). There was a significant reduction in their conjugation efficiency (e.g., 10⁴ fold). However, the non-dividing cells lyophilized using the un-optimized condition were shown to maintain their conjugation competency.

FIG. 9 shows a result demonstrating mobilization of several conjugative plasmids from non-dividing cells. The non-dividing cells were prepared from an E. coli strain S17-1 carrying a self-transmissible plasmid, F′ or R6Kdrd1. These non-dividing cells were mixed with a recipient E. coli strain, and spotted on a filter paper to facilitate conjugation. After one hour incubation at 37 C, the cell mixture was re-suspended in 0.9% NaCl. The cells were serially diluted, and grown on a LB plates containing appropriate antibiotics to selectively grow exconjugants. The numbers of growing colonies on the plate were used to calculate the efficiencies of conjugation. The RK2 plasmid was used as a positive control for the experiment. The results demonstrate that plasmids F′ and R6Kdrd1 are successfully mobilized from the non-dividing cells to the recipient bacterium.

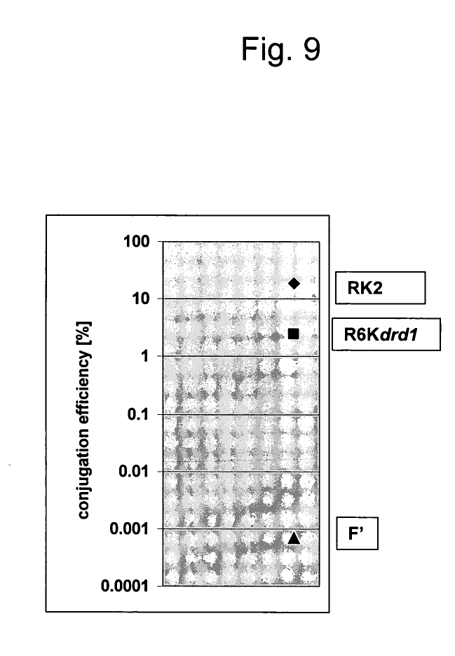

FIG. 10 shows the results of treating E. coli 0157:H7 with non-dividing cells on a flower surface. Panel 1 shows a plate spotted with serial dilutions of exconjugants. Panel 2 shows a Table comparing the number of exconjugants counted from the plate in Panel 1.

FIG. 11 shows the results of treating Salmonella enterica serotype Typhimurium (also known as Salmonella Typhimurium) with non-dividing cells on a flower surface. Panel 1 shows a plate spotted with serial dilutions of exconjugants. Panel 2 shows a Table comparing the number of exconjugants counted from the plate in Panel 1. “TNC” indicated that exconjugants were too numerous to count.

FIG. 12 shows the results of treating E. coli 0157:H7 with non-dividing cells on a leaf surface. Panel 1 shows a plate spotted with serial dilutions of exconjugants. Panel 2 shows a Table comparing the number of exconjugants counted from the plate in Panel 1. Panel 3 shows a graph comparing the survival values after conjugation from the data shown in the Table.

FIG. 13 shows the results of treating Salmonella enterica serotype Typhimurium with non-dividing cells on a leaf surface. Panel 1 shows a plate spotted with serial dilutions of exconjugants. Panel 2 shows a Table comparing the number of exconjugants counted from the plate in Panel 1. “TNC” indicated that exconjugants were too numerous to count. Panel 3 shows a graph comparing the survival values after conjugation from the data shown in the Table.

FIG. 14 shows the results of treating E. coli 0157:H7 with non-dividing cells on the surface of a potato. Panel 1 shows a plate spotted with serial dilutions of exconjugants. Panel 2 shows a Table comparing the number of exconjugants counted from the plate in Panel 1. “TNC” indicated that exconjugants were too numerous to count.

FIG. 15 shows the results of treating Salmonella enterica serotype Typhimurium with non-dividing cells on the surface of a potato. Panel 1 shows a plate spotted with serial dilutions of exconjugants. Panel 2 shows a Table comparing the number of exconjugants counted from the plate in Panel 1. “TNC” indicated that exconjugants were too numerous to count. Panel 3 shows a graph comparing the survival values after conjugation from the data shown in the Table.

FIG. 16 shows the results of treating E. coli 0157:H7 with non-dividing cells on the surface of meat. Panel 1 shows a plate spotted with serial dilutions of exconjugants. Panel 2 shows a Table comparing the number of exconjugants counted from the plate in Panel 1.

FIG. 17 shows the results of treating Salmonella enterica serotype Typhimurium with non-dividing cells on the surface of meat. Panel 1 shows a plate spotted with serial dilutions of exconjugants. Panel 2 shows a Table comparing the number of exconjugants counted from the plate in Panel 1.

FIG. 18 shows the results of treating E. coli 0157:H7 with non-dividing cells in blood plasma. Panel 1 shows a plate spotted with serial dilutions of exconjugants. Panel 2 shows a Table comparing the number of exconjugants counted from the plate in Panel 1. “TNC” indicated that exconjugants were too numerous to count.

FIG. 19 shows the results of treating Salmonella enterica serotype Typhimurium with non-dividing cells in blood plasma. Panel 1 shows a plate spotted with serial dilutions of exconjugants. Panel 2 shows a Table comparing the number of exconjugants counted from the plate in Panel 1. “TNC” indicated that exconjugants were too numerous to count. Panel 3 shows a graph comparing the survival values after conjugation from the data shown in the Table.

### DEFINITIONS

To facilitate an understanding of the invention, a number of terms are defined below.

As used herein, the term “nucleotide” refers to a monomeric unit of nucleic acid (e.g. DNA or RNA) consisting of a sugar moiety (pentose), a phosphate group, and a nitrogenous heterocyclic base. The base is linked to the sugar moiety via the glycosidic carbon (1′ carbon of the pentose) and that combination of base and sugar is called a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence” or “nucleic acid sequence,” and is represented herein by a formula whose left to right orientation is in the conventional direction of 5′-terminus to 3′-terminus.

As used herein, the term “base pair” refers to the hydrogen bonded nucleotides of, for example, adenine (A) with thymine (T), or of cytosine (C) with guanine (G) in a double-stranded DNA molecule. In RNA, uracil (U) is substituted for thymine. This term base pair is also used generally as a unit of measure for DNA length. Base pairs are said to be “complementary” when their component bases pair up normally by hydrogen bonding, such as when a DNA or RNA molecule adopts a double-stranded configuration.

DNA molecules are said to have “5′ ends” and “3′ ends” because mononucleotides are joined to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. A double-stranded nucleic acid molecule may also be said to have a 5′ and 3′ end, wherein the “5′” refers to the end containing the accepted beginning of the particular region, gene, or structure. A nucleic acid sequence, even if internal to a larger oligonucleotide, may also be said to have 5′ and 3′ ends (these ends are not ‘free’). In such a case, the 5′ and 3′ ends of the internal nucleic acid sequence refer to the 5′ and 3′ ends that said fragment would have were it isolated from the larger oligonucleotide. In either a linear or circular DNA molecule, discrete elements may be referred to as being “upstream” (or 5′) or “downstream” (or 3′) elements. Ends are said to “compatible” if a) they are both blunt or contain complementary single strand extensions (such as that created after digestion with a restriction endonuclease) and b) at least one of the ends contains a 5′ phosphate group. Compatible ends are therefore capable of being ligated by a double stranded DNA ligase (e.g. T4 DNA ligase) under standard conditions.

As used herein, the term “circular vector” refers to a closed circular nucleic acid sequence capable of replicating in a host.

As used herein, the terms “vector” or “plasmid” is used in reference to extra-chromosomal nucleic acid molecules capable of replication in a cell and to which an insert sequence can be operatively linked so as to bring about replication of the insert sequence. Examples include, but are not limited to, circular DNA molecules such as plasmids constructs, phage constructs, cosmid vectors, etc., as well as linear nucleic acid constructs (e.g., lambda phage constructs, bacterial artificial chromosomes (BACs), etc.). A vector may include expression signals such as a promoter and/or a terminator, a selectable marker such as a gene conferring resistance to an antibiotic, and one or more restriction sites into which insert sequences can be cloned.

As used herein, the terms “polylinker” or “multiple cloning site” refer to a cluster of restriction enzyme sites on a nucleic acid construct, which are utilized for the insertion, and/or excision of nucleic acid sequences.

As used herein, the term “host cell” refers to any cell that can be transformed with heterologous DNA (such as a vector). Examples of host cells include, but are not limited to, E. coli strains that contain the F or F′ factor (e.g., DH5αF or DH5αF′) or E. coli strains that lack the F or F′ factor (e.g. S17-1).

The terms “nucleic acid molecule encoding,” “DNA sequence encoding,” and “DNA encoding” refer to a sequence of nucleotides that, upon transcription into RNA and subsequent translation into protein, would lead to the synthesis of a given peptide. These terms also refer to a sequence of nucleotides that upon transcription into RNA produce RNA having a non-coding function (e.g., a ribosomal or transfer RNA). Such transcription and translation may actually occur in vitro or in vivo, or it may be strictly theoretical, based on the standard genetic code.

The term “gene” refers to a nucleic acid (e.g., DNA) sequence that comprises coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends for a distance of about 1 kb or more on either end, such that the gene is capable of being transcribed into a full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “expression” as used herein is intended to mean the transcription (e.g. from a gene) and, in some cases, translation to gene product. In the process of expression, a DNA chain coding for the sequence of gene product is first transcribed to a complementary RNA, which is often a messenger RNA, and, in some cases, the transcribed messenger RNA is then translated into the gene protein product.

As used herein, the term “toxic protein” refers to a protein that results in cell death or inhibits cell growth when expressed in a host cell.

As used herein, the term “toxic RNA” refers to an RNA that results in cell death or inhibits cell growth when expressed, e.g., in a target recipient cell.

As used herein, the term “toxic metabolite” refers to a metabolic product (e.g., of an enzyme reaction) that results in cell death or inhibits cell growth when the protein is expressed, e.g., in a target recipient cell.

As used herein, the term “replicable vector” means a vector that is capable of replicating in a host cell.

The term “expression vector” as used herein refers to a recombinant DNA molecule containing a desired coding sequence and appropriate nucleic acid sequences necessary for expression of the operably linked coding sequence (e.g. insert sequence that codes for a product) in a particular host organism. Nucleic acid sequences necessary for expression in prokaryotes usually include a promoter, an operator (optional), and a ribosome binding site, often along with other sequences such as a transcription terminator.

As used herein, the terms “restriction endonucleases” and “restriction enzymes” refer to enzymes (e.g. bacterial), each of which cut double-stranded DNA at or near a specific nucleotide sequence. Examples include, but are not limited to, Avail, BamHI, EcoRI, HindIII, HincII, NcoI, SmaI, and RsaI.

As used herein, the term “restriction” refers to cleavage of DNA by a restriction enzyme at its restriction site.

As used herein, the term “restriction site” refers to a particular DNA sequence recognized by its cognate restriction endonuclease.

As used herein, the term “purified” or “to purify” refers to the removal of contaminants from a sample. For example, plasmids are grown in bacterial host cells and the plasmids are purified by the removal of host cell proteins, bacterial genomic DNA, and other contaminants. Thus the percent of plasmid DNA is thereby increased in the sample. In the case of nucleic acid sequences, “purify” refers to isolation of the individual nucleic acid sequences from each other.

As used herein, the term “PCR” refers to the polymerase chain reaction method of enzymatically amplifying a region of DNA. This exponential amplification procedure is based on repeated cycles of denaturation, oligonucleotide primer annealing, and primer extension by a DNA polymerizing agent such as a thermostable DNA polymerase (e.g. the Taq or Tfl DNA polymerase enzymes isolated from Thermus aquaticus or Thermus flavus, respectively).

As used herein, the terms “complementary” or “complementarity” are used in reference to polynucleotides (e.g., a sequence of nucleotides) related by the base-pairing rules. For example, for the sequence “5′-A-G-T-3′,” is complementary to the sequence “3′-T-C-A-5′” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

As used herein, the term “oligonucleotide,” refers to a short length of single-stranded polynucleotide chain. Oligonucleotides are typically less than 100 residues long (e.g., between 15 and 50), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The term “transformation” or “transfection” as used herein refers to the introduction of foreign DNA into cells (e.g. prokaryotic cells). Transformation may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The terms “microorganism” and “microbe” may be used interchangeably; as used herein these terms mean an organism too small to be observed with the unaided eye and includes, but is not limited to bacteria, virus, protozoans, fungi, and ciliates.

The term “microbial gene sequences” refers to gene sequences derived from a microorganism.

The term “bacteria” refers to any bacterial species including eubacterial and archaebacterial species.

The term “virus” refers to obligate, ultramicroscopic, intracellular parasites incapable of autonomous replication (e.g., replication requires the use of the host cell's machinery).

As used herein, the term “conjugation” refers to the process of DNA transfer from one cell to another. Although conjugation is observed primarily between bacterial cells, this process takes place from bacterial cells to higher and lower eukaryote (Waters, Nat Genet. 29:375-376 [2001]; Nishikawa et al., Jpn J Genet. 65:323-334 [1990]). Conjugation is mediated by complex cellular machinery, and essential protein components are often encoded as a series of genes in a plasmid (e.g., the tra genes for RK2). Some of these gene products are assembled to facilitate a direct cell-cell interaction (e.g., mating pair formation), and some of them serve to transfer DNA and associated protein molecule and to replicate the DNA molecule (e.g., DNA transfer/replication). oriT is a DNA sequence, where the transfer of a DNA molecule initiates in the process of conjugation.

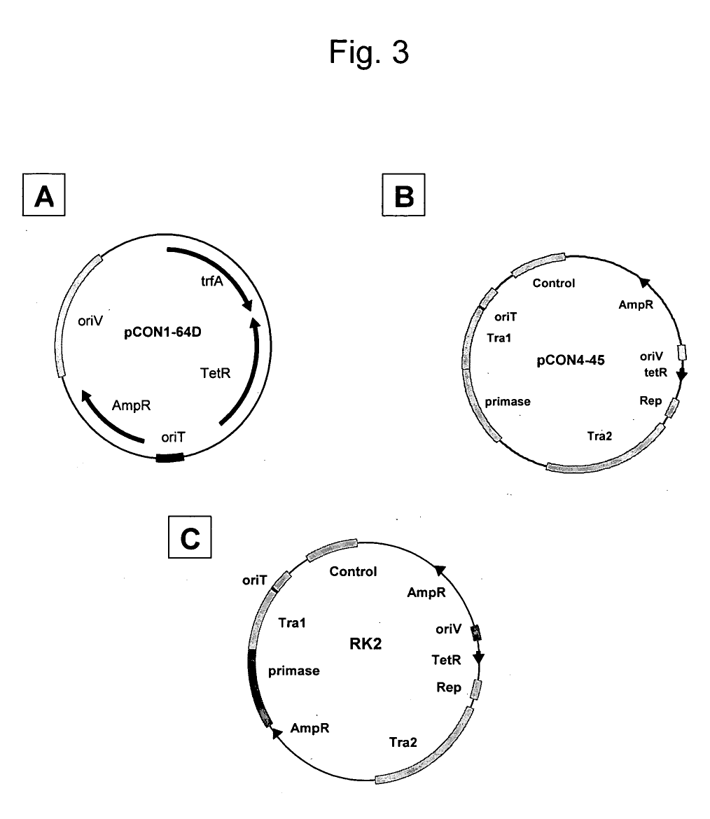

As used herein, the terms “conjugation donor” and “donor cell” are used interchangeably to refer to a cell, generally a bacterial cell, carrying a plasmid, wherein said plasmid can be transferred to another cell through conjugation. Example of donor cells include, but are not limited to E. coli strains that contain a self-transmissible plasmid (e.g., F, F′, RK2, R6K) or a non-self-transmissible plasmid (e.g., pCON1-64d in FIG. 3C). A cell receiving a plasmid or other cellular material from a donor cell via conjugative transfer is referred to as a “recipient cell”. As used herein, the term “transmissible plasmid” refers to a plasmid that can be transferred from a donor cell to a recipient cell via conjugation.

As used herein, the term “self-transmissible plasmid” refers to a plasmid encoding all the genes needed to mediate conjugation (e.g., RK2, F and R6K). A recipient of a self-transmissible plasmid becomes a proficient donor to further transfer the self-transmissible plasmid to another recipient cell.

As used herein, the term “non-self-transmissible plasmid” or “mobilizable plasmid” refers to a plasmid lacking some of the genes needed to mediate conjugation. A cell carrying a non-self-transmissible plasmid does not transfer DNA through conjugation unless the missing gene(s) are supplied in trans within the same cell. Therefore, a recipient cell that lacks the missing gene(s), does not become a proficient conjugation donor when it receives the non-self-transmissible plasmid.

As used herein, the term “origin of transfer” or “oriT” refers to the cis-acting site required for DNA transfer, and integration of an oriT sequence into a non-transmissible plasmid converts it into a mobilizable plasmid (Lanka and Wilkins, Annu Rev Biochem, 64:141-169 [1995]).

As used herein, the term “maxicell” refers to the UV light irradiated cells that have been further treated, e.g., by extended incubation after irradiation, to maximize chromosomal degradation. Maxicells contain mostly plasmid DNA, and synthesis of proteins within maxicells occurs essentially exclusively from the plasmid DNA in the cells.

As used herein, the term “non-dividing cell” or “ND cell” refers to cells that are treated in a manner selected to preferentially damage the chromosomal DNA of the cell (e.g., by UV or other irradiation), wherein said cells are further treated, e.g., by rapid chilling after DNA damaging treatment, to minimize chromosomal degradation. ND cells contain both chromosomal and plasmid DNA but the chromosomal DNA is sufficiently altered by UV irradiation that said ND cells have little or no capability to divide.

As used herein, the term “cryopreservative” refers to a chemical element within the medium to prevent cellular damages caused by ice formation in both outside and inside of the cell upon freezing. These cryopreservatives include, but not limited to, glycerol, sucrose, trehalose, DMSO and ethylene glycol.

As used herein, the term “kit” refers to any delivery system for delivering materials. In the context of reaction materials such as non-dividing cells, such delivery systems include systems that allow for the storage, transport, or delivery of reaction reagents (e.g., cells, buffers, selection reagents, etc., in the appropriate containers) and/or supporting materials (e.g., media, written instructions for performing using the materials, etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain cells for a particular use, while a second container contains selective media. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction materials needed for a particular use in a single container (e.g., in a single box housing each of the desired components). The term “kit” includes both fragmented and combined kits.

As used herein, the term “cellular metabolic function” refers to any or all processes conducted by a cell (e.g., enzymatic or chemical processes associated with cell function), other than genomic replication.

### DESCRIPTION OF THE INVENTION

The present invention provided non-dividing cells that are useful in the delivery of genetic material to target bacterial cells by conjugation. Bacterial conjugation as a means of delivering DNA or/and associated components into bacteria, e.g., pathogenic bacteria, to kill them at the site of infection is one application of the compositions and methods of the present invention (see, e.g., PCT Publication WO 02/18605, to Filutowicz, incorporated herein in its entirety). Such targeted bacterial killing also has agricultural and industrial uses, including but not limited to applications such as reducing the presence of bacteria or retarding the growth of bacteria in foods, in animal feeds, on live plants and cut flowers, and on industrial surfaces and in industrial materials. For such applications, it is highly desirable to use donor cells having very high conjugation efficiency, such as are provided by the methods and compositions of the present invention. In contrast to the aggressive treatments used to produce maxicells that dramatically reduce conjugation competence of the cells, the present invention provides methods of treating bacterial cells to make them non-dividing (i.e., non-viable), while preserving their cellular metabolic function. In particular, the methods of the present invention provide methods for producing non-dividing cells while preserving their conjugation competence. Such metabolically active non-dividing cells are very useful tools for the delivery of biomolecules, especially DNA, e.g., via conjugative transfer or excretion, and are equipped with the highest level of genetic containment so as to prevent the spread of genetically modified bacterial cells.

Bacterial conjugation is a major mode of gene transfer among bacterial species, and requires a direct cell-cell interaction. A large number of conjugative plasmids are known to the art, and they are classified based on incompatibility group (Inc). Members of each group represent the same or closely related conjugation system (Lanka and Wilkins, Annu Rev Biochem. 64:141-169 [1995]). Among these plasmids, RK2 is one of the best-studies broad-host range conjugative plasmid (Helinski et al., Escherichia coli and Salmonella: Cellular and Molecular Biology, 2:2295-2324, ASM press [1996]). RK2 is a self-transmissible plasmid belonging to the IncPα group, and its entire DNA sequence has been reported (Pansegrau et al., J. Mol. Biol. 239:623-663 [1994]). Along with genes essential for conjugation, RK2 encodes genes responsible for antibiotic resistance, segregational stability and vegetative replication of the plasmid. A plasmid can be conjugatively transferred by a helper-independent (self-transmissible) or a helper-dependent (non-self-transmissible) manner. A self-transmissible vector carries all the genes (tra genes) essential for conjugation: mating pair formation and DNA transfer/replication. A recipient of a self-transmissible plasmid becomes a proficient conjugation donor after receiving the plasmid. In contrast, a non-self-transmissible plasmid can replicate but lacks genes to establish the conjugation machinery. However, a non-self-transmissible plasmid can be conjugatively transferred only when functional tra gene products are provided in trans in the same bacterial cell. Such tra genes can be encoded on another plasmid or the host chromosome (Simon et al., Bio/Technology 1:784-791 [1985]; Giebelhaus et al., J. Bacteriol., 178:6378-6381 [1996]). The conjugation machinery is very complex, and tightly associated with cytoplasmic membrane, and its process requires energy (e.g., ATP). Conjugal DNA transfer mediated by RK2 is very efficient, which often reaches to the efficiency of 100%.

Bacterial conjugation requires metabolic functions of the donor bacteria, the bacterial ghosts described above cannot be used since the ghosts lacks majority of cellular metabolism. Minicells retain the cellular metabolic functions, including some capacity of bacterial conjugation (Frazer and Curtiss III, Curr. Top. Microbiol. Immunol., 69:1-84 [1975]). However, preparation of minicells is time consuming, and minicells have been determined to have a conjugation efficiency that is significantly lower than that of live bacterial donors cells (Frazer and Curtiss III, Curr. Top. Microbiol. Immunol., 69:1-84 [1975]). As described above, the harsh treatment used to damage and degrade the bacterial chromosomal DNA during the production of maxicells damages cellular metabolic function, and significantly reduces the conjugation competency of these cells.

In some embodiments, the present invention provides a method to generate conjugation-competent non-dividing cells. In some embodiments, this method utilizes combinations of bacterial mutants lacking one or a group of gene functions responsible for DNA repair after DNA-damaging irradiation such as is used in the production of maxicells. Unlike maxicells, however, the non-dividing cells of the present invention are not treated to degrade the damaged chromosomal DNA.

The systems, method and compositions of the present invention are laid out in the following sections: I. Bacterial Hosts; II. Generation of Non-Dividing Cells with Preserved Metabolic Function; III. Storage of Non-Dividing Cells. IV. An Application of the Methods and Compositions of the Present Invention; V. Kits.

I. Bacterial Hosts

In some embodiments, the present invention provides examples of hosts harboring specific mutations. As used herein, the term “bacterial host” refers to a bacterium that, upon transformation with a transmissible plasmid, becomes a donor cell. In preferred embodiments, the bacterial hosts carry the recA mutation. The present invention is not limited to the use of only the recA mutation. Since a large number of mutations involved in the DNA repair system besides recA are known to the art (Kuzminov, Microbiol Mol Biol Rev, 63:751-813 [1999]; Carell et al., Curr Op Chem Biol 5:491-498 [2001]), one would use a bacterial strain carrying one or a group of these mutations in addition to the recA mutation.

In some embodiments, E. coli is used as a bacterial host. The present invention is not limited to E. coli, since the DNA repair systems among bacteria share a close similarity (Kuzminov, Microbiol Mol Biol Rev, 63:751-813 [1999]; Petit and Sancar, Biochimie, 81:15-25 [1999]; Yasui and McCready, Bioessays, 20:291-297 [1998]). Therefore, one may use either a Gram-negative or Gram-positive bacterium.

In some embodiments, the conjugation system from RK2 is used to transfer a plasmid from a bacterial host. The present invention is not limited to utilize the RK2 conjugation system. Since a number of conjugation systems and their close similarity are known to the art (Lanka and Wilkins, Annu Rev Biochem. 64:141-169 [1995]; Grohmann et al. Micoriol Mol Biol Rev. 67: 277-301 [2003]), one or a group of these systems may be combined.

In some embodiments, tra genes encoding essential components for conjugation machinery are used. Locations of the tra genes are not limited to a plasmid. The tra genes can be located on the host chromosome (Simon et al, Bio/Technology 1:784-791 [1985]). The tra genes can be located on a plasmid itself. The tra genes may be located in both the host chromosome and a plasmid.

II Generation of Non-Dividing Cells with Preserved Metabolic Function

The present invention provides methods and compositions for the generation of non-dividing bacterial cells, wherein said non-dividing bacterial cells retain an ability to conjugate efficiently. In some embodiments, said bacterial cells comprise one or a group of mutations in DNA repair system.

In some embodiments UV light is used to introduce DNA damage within the host bacterial cells. The present invention is not limited to UV light to introduce damage, since other irradiation is also known to the art for causing DNA damage (Paskvan et al., FEMS Bicrobiol Lett. 205:299-303 [2001]; Kogoma et al. J. Bacteriol. 178:1258-1264). In preferred embodiments, the cells are treated after irradiation so as to reduce degradation of the damaged DNA. In particularly preferred embodiments, irradiated cells are immediately transferred to a cold temperature after such DNA-damaging irradiation to produce conjugation-competent non-dividing bacterial cells, wherein said conjugation-competent non-dividing bacterial cells are useful to deliver DNA or/and associated protein into pathogenic bacteria to control the pathogenic bacteria at a site of infection (Heinemann, Plasmid 41:240-247 [1999]; Ziemienowicz et al., Proc Natl Acad. Sci., 96:3729-3733 [1999]). In more particularly preferred embodiments, cold temperature (e.g., about 0° to about 10° C.) is used to treat the cells immediately after UV light-irradiation. In still more particularly preferred embodiments, an ice-cold temperature (e.g., about 0° to about 5° C.) is used. This step is not limited to the use of ice-cold temperature. Since we also demonstrated that these non-dividing cells were stably maintained at a lower temperature such as between −80° C. and 0° C., in some embodiments one may transfer the UV light-irradiated bacteria directly into a freezing (e.g., below 0° C.) environment immediately after the irradiation. It is also known to the art that lower temperatures preserve frozen cells (Hubalek, Cryobiol., 46:205-229 [2003]) therefore, one may use other means of cooling (e.g., dry ice and liquid nitrogen) after the DNA-damaging irradiation.

Testing Conjugation Competency

Non-dividing cells were prepared from either JM109 or S17-1 carrying a self-transmissible plasmid pCON4-45 (FIG. 3). Both the above strains are derivatives of E. coli K12, and their relevant genotype is recA deficient; therefore, the UV-damaged DNAs can not be repaired efficiently. These plasmid-harboring bacterial donors were irradiated with UV, and used as conjugation-competent non-dividing cells. These non-dividing cells were tested for their conjugation competency using another E. coli strain. RK2, one of the best-studied conjugative plasmid, was used to demonstrate that this plasmid could efficiently mobilize from the non-dividing-cells to recipient bacterial cells.

Testing Conjugation Between Non-Dividing Cells and Selected Recipient Cells

Conjugation is a bacterial mating, and a large number of conjugative plasmids are known to the art. Of these, RK2 is one of the best-studied broad-host-range plasmids. This plasmid can replicate and conjugate in almost all gram-negative bacteria. Conjugation takes place in both.intra- and inter-species manners; therefore, a common laboratory bacterium such as E. coli has the ability to conjugate a broad range of recipient strains including, but not limited to Salmonella, Pseudomonas, Klebsiella, and Proteus. Therefore, it is generally convenient to conduct initial tests on a target cell using a broad-host-range conjugative plasmid such as RK2, and E. coli as a host bacterial strain. If E. coli does not conjugate well with the target strain, or if the target bacterium resides within an environment too harsh for E. coli-non-dividing cells to survive (e.g., pH, temperature, osmolarity and defense mechanism from an animal host), one would prepare conjugation competent non-dividing cells using the targeted strain, or closely-related bacterial strain. Some of these bacterial strains are listed in the table below. A number of conjugative plasmids maintained in these strains are known to the art (Waters, Frontiers Biosci, 4:d416-439 [1999]; Hofreuter and Haas, J. Bacteriol, 184:2755-2766 [2002]; Leaves et al., J Antimicrob Chemother, 45:599-604 [2000]; Alvarez et al., Antimicrob Agent Chemother 48; 533-537 [2004]; Grohmann et al., Microbiol Mol Biol Rev, 67:277-301 [2003]). In addition, the genome sequence of the listed bacteria are completed except K. pneumoniae, which one would use to design experiments to modify their genomes. Additional examples of suitable strains for non-dividing cell preparation include various species of Lactobacillus (such as L. casei, L. plantarum, L. paracasei, L. acidophilus, L. fermentum, L. zeae and L. gasseri), and other nonpathogenic or probiotic skin-or GI colonizing bacteria such as Lactococcus and Bifidobacteria. Additional examples of Gram negative and Gram-positive bacteria are provided below, in Table 1. Preparation and testing of conjugation competent non-dividing cells from the target organism can be conducted as described in Example 6.

TABLE 1
Examples of bacteria useful for non-dividing cell production

| | Example applications |
|---|---|
| Gram-negative bacteria | |
| Helicobacter pylori | stomach ulcer and cancer |
| Pseudomonas aeruginosa | infection at lung, urinary tract, burn |
| Haemophilus influenzae, | influenza |
| somnus and ducreyi | |
| Klebsiella pneumoniae | infection at urinary tract and lung |
| Gram-positive bacteria | |
| Lactococcus lactis | processing dairy products |
| Lactobacillus plantarum | probiotic to control bacterial flora in an |
| | intestinal system |
| Bacillus subtilis | industrial use for enzyme secretion |
| Staphylococcus species | infection at many different sites |
| Streptococcus species | infection at many different sites |

III Storage of Non-Dividing Cells

In some embodiments the present invention provides examples of methods for storage of conjugation-competent non-dividing bacterial cells for extended periods of time. The present invention is very useful to store and deliver the conjugation-competent non-dividing bacterial cells to a location and a time when it is required.

In some embodiments, glycerol is used as cryopreservative. Other cryopreservatives may be utilized, including but not limited to trehalose, sucrose, DMSO and ethylene glycol (Hubalek, Cryobiol., 46:205-229 [2003]).

In additional embodiments, lyophilization is used by means of storage. Lyophilization is very useful to store and deliver the conjugation-competent non-dividing bacterial cells where a storage system is not available at a very low temperature (e.g., −80° C. freezer).

In additional embodiments, dry ice-ethanol is used to as an example of method to freeze the conjugation-competent non-dividing bacterial cells. The method is not limited to dry ice-ethanol. Other methods of freezing include but are not limited to liquid nitrogen and dry ice.

IV Application of the Methods and Compositions of the Present Invention

The methods of the present invention provide generation and storage of conjugation-competent non-dividing bacterial cells. As shown here and as further shown in the Examples, one exemplary application of the the non-dividing cells of the present invention is in the conjugation-mediated killing of pathogenic bacteria.

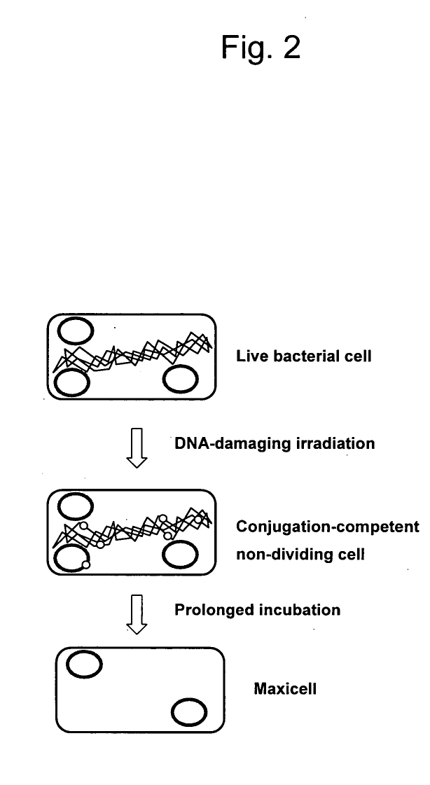

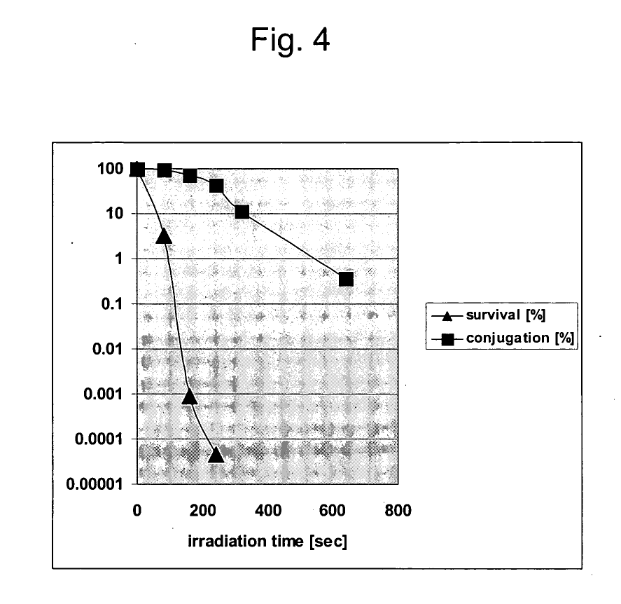

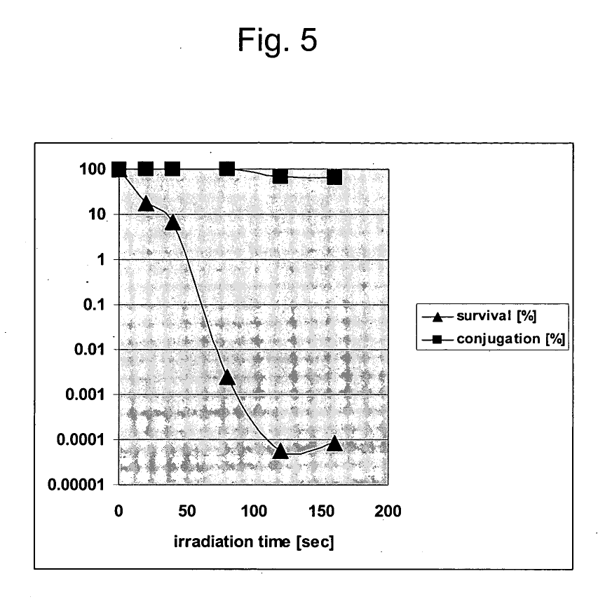

FIG. 2 describes an exemplary schema to generate conjugation-competent non-dividing bacterial cells. Two recA mutants were used for these experiments (JM109 and S17-1). JM109 and S17-1 were used to carry a self-transmissible plasmid RK2 and non-self-transmissible plasmid pCON1-64D, respectively (FIGS. 3A and B), and conjugation-competent non-dividing bacterial cells were prepared from both strains (FIGS. 4 and 5). The viabilities of these bacterial strains dropped sharply upon UV light-irradiation; however, the efficiencies of their conjugation remained high even after the viability dropped below 0.001%. RecA is not the only enzyme involved in DNA repair system in bacterial cells (Kuzminov Microb Mol Biol Rev. 63:751-813 [1999]0, additional mutation including, but not limited to, phrB (gene encoding photolyase) and uvrA (gene encoding a subunit of exonuclease) would improve the process of generating non-dividing cells. In this study, RK2 was used as a conjugal plasmid to provide the conjugation machinery in the bacterial cells, and both self- and non-self transmissible plasmids were shown to conjugate efficiently from the conjugation-competent non-dividing cells. Since bacterial conjugation systems share a high level of similarity, another conjugation system besides RK2 would be easily used to generate the conjugation-competent non-dividing bacterial cells. Along with the conjugation systems, the DNA repair systems in bacteria share also close similarities, mutations in the repair system can be introduced in a wide range of bacterial species (Petit and Sancar, Biochemie, 81:15-25 [1999]; Yasui and McCready, Bioessays, 20:291-297 [1998]). All together, generation of conjugation-competent non-dividing bacterial cells can be prepared from a large number of bacteria belonging both Gram-negative and Gram-positive bacteria (Eitner et al., Mol Gen Genet., 195:516-522 [1984]; van Waasbergen et al., Appl Microbiol Biotechnol., 49:59-65 [1998]; Shibata and Ando Mutat Res., 30:177-190 [1975]; Mikoc et al., Mol Gen Genet., 264:227-232 [2000]). The generated conjugation-competent non-dividing bacterial cells can be used to deliver DNA or associated elements into target bacteria with a genetic containment system to prevent the spread of the donor bacteria (Heinemann, Plasmid 41:240-247 [1999]; Ziemienowicz et al., Proc Natl Acad. Sci., 96:3729-3733 [1999]). Any useful genes can be cloned into the delivered DNA, and such genes can be expressed in the target bacteria. Such genes may encode, but are not limited to, a bactericidal protein such as a colicin. A tightly-controlled promoter represses such a bactericidal gene in a donor bacterial cell (i.e., conjugation-competent non-dividing bacterial cell), and the gene expresses the toxic product in the target cell where the repression is removed. Therefore, as an example, conjugation-competent non-dividing bacterial cells carrying a bactericidal gene can be used to kill unwanted bacteria at the site of infection.

As described above, a variety of methods to store live bacteria are known to the art (Hubalek, Cryobiol., 46:205-229 [2003]). In this study we demonstrated that conjugation-competent non-dividing bacterial cells can be stored extended periods of time. When bacteria are stored in a frozen form, cryopreservatives are used to reduce cellular damages caused by osmotic imbalance and crystallization of intercellular water. A number of cryopreservatives are known to the art including, but not limited to, trehalose, glycerol, sucrose, DMSO and ethylene glycol. We used glycerol as an example to demonstrate the feasibility of storing conjugation-competent non-dividing bacterial cells. As described in Example 5, UV light-irradiated non-dividing cells were demonstrated that they retain their conjugation efficiency at least up to 3 months in −80 C. It is suggestive that if these cells were stored stably up to 3 months, it is very likely that they would be stored for even more extended periods of time (e.g., 6 months, 1 year, 5 years, 10 years).

In addition to cryopreservation, lyophilization is also known to the art as a method to preserve live cells (ATCC, FAQs). Since lyophilized cells can be stored at relatively higher temperature compared to frozen cells, this storage method will provide flexibility in shipping and storage of the conjugation-competent non-dividing cells.

V. Kits

In some embodiments, the present invention provides kits comprising non-dividing bacterial cells carrying a transmissible element of interest. In some embodiments the non-dividing cells are frozen, while in other embodiments, the non-dividing cells are dried. In some embodiments, the non-dividing cells in a kit are formulated in tablets, capsules, or as a powder. In some embodiments, a kit may further comprise buffers. In still other embodiments, a kit may comprise written materials related to the purpose or use of the kit materials.

### EXPERIMENTAL

The following examples are provided in order to demonstrate and further illustrate certain embodiments and aspects of the present invention and are not to be construed as limiting the scope thereof.

### Example 1

### Plasmid Construction

This example describes the construction of exemplary plasmids to for use in the methods and compositions of the present invention.

A. Materials and Methods

Bacterial Strains and Media

The Escherichia coli strain utilized was JM109 [F′ traD36 proA⁺B⁺ lacl^{q} Δ (lacZ) M15/Δ(lac-proAB) glnV44 el4⁻ gyrA96 recA1 relA1 endA1 hsdR17]. All cloning was performed using standard methods known in the art, and using Luria Bertani growth media supplemented with 50 μg/ml kanamycin and/or 15 μg/ml tetracycline and/or 100 μg/ml ampicillin to permit selection for plasmids.

B. Plasmid Construction

Construction of pCON4-45

A DNA adaptor comprising multiple cloning sites (NsiI-MluI-NheI-SacI-AsiSI) was made by hybridizing a pair of DNA oligomers (5′ TACGCGTGCTAGCGAGCTCATTAATGCGAT 3′, SEQ ID NO:1, and 5′ CGCATTAATGAGCTCGCTAGCACGCGTATGCA3′, SEQ ID NO:2). The duplex adapter was cloned into the NsiI-AsiSI site of natural plasmid RK2 (diagrammed in FIG. 3C; Pansegrau et al., M. Mol. Biolo. 239:623-663 [1994]) by replacing the 6 kb NsiI-AsiSI region to generate pCON4-45 (see FIG. 3B). Part of this 6 kb region contains a kanamycin-resistance determinant, therefore the generated plasmid, pCON4-45, is resistant to only tetracycline and ampicillin.

Construction of pCON1-64d

The tetracycline-resistance determinant was cloned from RK2 by PCR using a pair of primers (5′GGTCGACTATCGTTTCCACGATCAGCGAT3′, SEQ ID NO:3, and 5′CAAGCTTGGATCACTGTATTCGGCTGCAA3′, SEQ ID NO:4) to amplify this fragment using standard PCR conditions. The PCR-amplified fragment was cloned into the HindIII-NsiI site of a mini-RK2 plasmid called pRR10 (Roberts et al., J. Bacteriol., 172:6204-6216 [1990]) to generate pCON1-64d (see FIG. 3A). This plasmid carries two antibiotic selective markers (ampR for ampicillin; tetA for tetracycline); oriT, origin of transfer; oriV; origin of replication and trfA; a gene encoding replication initiator, which is essential for replication from oriV.

### Example 2

### Bacterial Conjugation

This example provides an exemplary description of a method to perform bacterial conjugation.

A. Materials and Methods

Bacterial Strains and Media

The Escherichia coli strains utilized were S17-1 (Simon et al, Bio/Technology 1:784-791 [1985]), JM109 (Yanish-Perron et al., Gene 33:103-119 [1985]) and RL315. S17-1 is a K12-derived, non-pathogenic strain which is widely used in research laboratories working on bacterial conjugation. This strain carries the entire set of tra genes (derived from RK2) integrated into its chromosome, whose expression facilitates conjugal transfer of a resident mobilizable plasmid to a recipient. JM109 is also a K12-derived strain, and its relevant genotype is recA minus. S17-1 is also recA deficient, which harnesses the process of DNA-repair function after DNA-damaging irradiation. S17-1 was utilized to generate conjugation-competent non-dividing cells. Another K12-derived E. coli strain RL315 was utilized as a recipient strain of conjugation. Rifampicin resistance of RL315 was used to select this strain, and counter-select a conjugation donor strain, which is sensitive to this antibiotic. Both strains were cultured in LB medium overnight at 37 C with agitation, and the cell densities were adjusted to 1.0 at OD600 in prior to experiments.

B. Bacterial Conjugation

Donor and target cells were grown overnight in LB medium containing appropriate antibiotics, and adjusted to OD₆₀₀ 1.0 prior to a conjugation experiment. The same amount of donor and recipient cells were mixed together and spun down. The cell pellets were re-suspended in a small volume of 0.9% NaCl, spotted on a nitrocellulose filter, and incubated on an LB plate for 1 hour without any antibiotic selection. After the incubation, cells were re-suspended in 0.9% of NaCl, serially diluted, and spotted on LB-antibiotic plates for measuring colony forming units (cfu). Exconjugants were selected by both selection markers, rifamipicin-resistance (RifR) and tetracycline-resistance (TetR), which should prevent growth of donor and target bacteria in the mixed cell suspension. LB plates containing Rif were used to calculate the total number of target cells. pCON4-45 was conjugatively transferred into RL315 using S17-1 as the donor strain, and exconjugants were serially diluted for plating on Rif and Rif/Tet plates. Efficiencies of conjugation were calculated using the equation shown below.

$$\frac{number of colonies on Rif / Tet per unit volume}{number of colonies on Rif per unit volume} \times 100 = conjugation efficiency [ \% ]$$

### Example 3

### Conjugal Transfer of a Self-Transmissible Plasmid from Non-Dividing JM109

This example demonstrates the transfer of a self-transmissible plasmid, RK2, from a non-dividing cell to a recipient cell, and shows that JM109 is a suitable strain for generating non-dividing cells using the method described in Example 3.

E. coli strain JM109 carrying the recA mutation was grown overnight in LB medium containing appropriate antibiotics. The cells were spun down, re-suspended in 0.9% NaCl and adjusted to OD₆₀₀ 1.0 prior to UV irradiation. Ten to fifteen milliliters of the cell suspension was transferred into a Petri dish, which was placed on a rotary shaker. A UV illuminator [302 nm] was placed inverted above the rotary shaker. The intensity of UV and the distance between the surface of the cell suspension and the UV lamp were kept constant. The cell suspensions were exposed to the UV light on the rotary shaker at 60 rpm, and bacterial cells were collected at different dosages of UV irradiation. The cells were immediately transferred onto ice after UV irradiation. These collected cells were serially diluted and spread on LB plates to monitor their viability by measuring colony forming units.

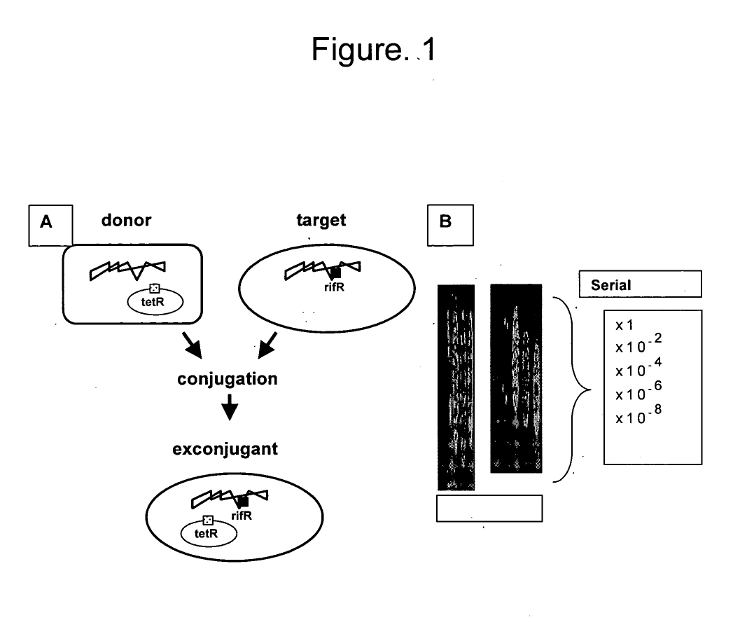

A self-transmissible plasmid, wild-type RK2, was tested for its conjugal transfer from a non-dividing cell prepared from a laboratory E. coli strain JM109 as described in Example 3. The entire tra genes essential for conjugation are located on RK2, therefore all the key components essential for the conjugation machinery were synthesized from the plasmid-encoded genes. JM109 cells harboring RK2 were irradiated with variable amounts of UV dosage, and further used to test their conjugation capability using an E. coli strain RL315 as a recipient bacterium. Viabilities of UV-irradiated bacteria were monitored by counting colony forming units on a LB plate containing 15 ug/ml of tetracycline. Conjugation was carried out using a regular filter conjugation (see FIG. 1), and exconjugants were selected on LB plates containing both rifampicin and tetracycline.

As summarized in FIG. 4, the UV-irradiated bacterial cells retain conjugation capacity at significant levels for extended UV dosages. For example, after 72 seconds of UV irradiation, the survival of bacteria drops to 0.00091%, but the efficiency of conjugation was retained at 72%. The results clearly demonstrate that bacterial cells can conjugate efficiently after the cells lose their viability.

### Example 4

### Conjugal Transfer of Non-Self-Transmissible Plasmid from Non-Dividing S17-1

This example describes that a non-self-transmissible plasmid mobilize to a recipient cell from a non-dividing cell using pCON1-64D as the conjugative plasmid as described in Example 1, and S17-1 as a source bacterial strain for generating non-dividing cells using the method described in Example 3.

E. coli strain S17-1 carrying the recA mutation was grown overnight in LB medium containing appropriate antibiotics The cells were spun down, re-suspended in 0.9% NaCl and adjusted to OD₆₀₀ 1.0 prior to UV irradiation. Ten to fifteen milliliter of the cell suspension was transferred into a Petri dish, which was place on a rotary shaker. A UV illuminator [302 nm] was placed inverted above the rotary shaker. The intensity of UV and the distance between the surface of the cell suspension and the UV lamp were kept constant. The cell suspensions were exposed to the UV light on the rotary shaker at 60 rpm, and bacterial cells were collected at different dosages of UV irradiation. The cells were immediately transferred onto ice after UV irradiation. These collected cells were serially diluted and spread on LB plates to monitor their viability by measuring colony forming units.

Since pCON1-64d lacks essential tra genes on its plasmid, the products of the tra genes essential for conjugation have to be provided from the chromosome of the host strain S17-1. S17-1 cells harboring pCON1-64d were irradiated with variable dosage of UV light, and further used to test their conjugation capability using an E. coli strain RL315 as a recipient bacterium. Conjugation was carried out using a regular filter conjugation (see Example 2), and exconjugants were selected on LB plates containing rifampicin and tetracycline. The results were summarized in FIG. 5.

As summarized in FIG. 5, the UV-irradiated bacterial cells retain conjugation capacity at significant levels for extended UV dosages. For example, after 80 seconds of UV irradiation, the survival of bacteria drops to 0.0024%, but the efficiency of conjugation was retained at 100%. The result clearly demonstrated that bacterial cells can conjugate efficiently after the cells lost their viability.

### Example 5

### Cryopreservation of Conjugation-Competent Non-Dividing Cells

Non-dividing cells were prepared from strain S17-1 carrying a non-self-transmissible plasmid pCON1-64D as described in Example 4. The UV-treated non-dividing cells were re-suspended in 0.9% NaCl containing 20% of glycerol. The cell suspension was quickly frozen using dry ice-ethanol bath, and stored in −80 C. The stored non-dividing cells were thawed after extended time periods, and their conjugation efficiencies were monitored using the method as describe in Example 2.

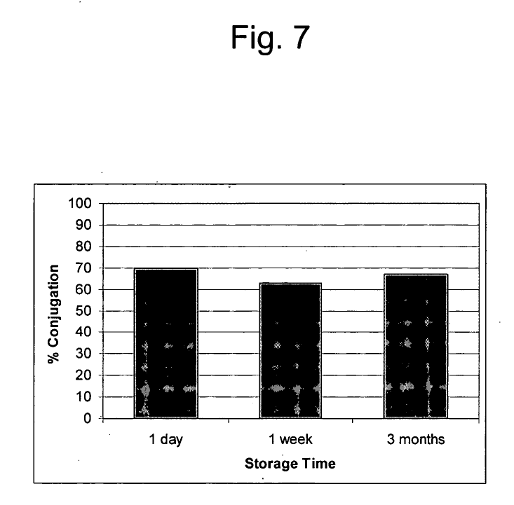

The result was summarized in FIG. 7. For this example, 120-second UV irradiation was applied, and the survival of the donor strain (S17-1 carrying pCON1-64D) was 0.004%. Although approximately 30% of reduction was observed in the conjugation efficiency upon freezing, the stored non-dividing cells retained their conjugation capacity without any significant change within the storage period we tested (up to about 3 months). The result was demonstrating that non-dividing cells can be stored in −80 C for extended period of time without significant changes in their conjugation efficiency, which is very important because the same batch of non-dividing cells can be supplied upon request. It should be also noted that non-dividing cells can be easily sent with a pack of dry ice.

### Example 6

### Lyophilization of Conjugation-Competent Non-Dividing Cells

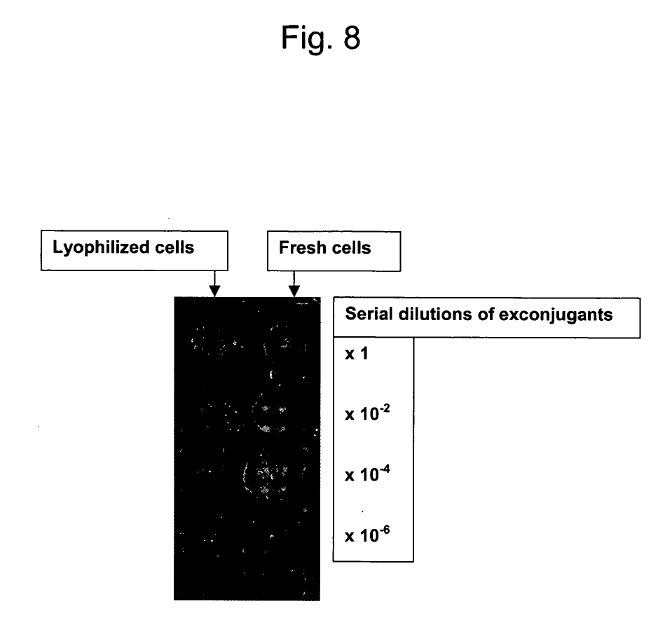

Non-dividing cells were prepared from S17-1 carrying a self-transmissible plasmid pCON4-45 (FIG. 3) as described in Example 3. The UV-treated non-dividing cells were re-suspended in 0.9% NaCl, and quickly frozen using dry ice-ethanol bath. The frozen cells were lyophilized, and stored at 4° C. Water was added to the lyophilized cells to regenerate the cells, and they were used for conjugation as described in Example 2. The results are shown in FIG. 8.

Although the efficiency was lower (about 0.001%) compared to that of freshly prepared non-dividing cells, we could demonstrate that the re-hydrated lyophilized non-dividing cells are capable of conjugation.

### Example 7

### General Method for Producing and Testing Non-Dividing Cells

The following provides a general method for the generation of non-dividing cells. The methods described below are provided as an example of a procedure for selecting cells for use in the methods and compositions for the invention. These exemplary methods are not intended to limit the invention to this or any other particular procedure for making and selecting non-dividing cells. In addition, these exemplary methods are provided as examples of a process for preparing non-viable cells then screening the prepared cells for survival of a particular desirable trait, such as a metabolic trait. The methods and compositions of the present invention are not limited to the production of conjugation competent cells.

The non-dividing cells of the present invention are generally derived from cells having an impaired ability to repair DNA, e.g., recA mutants. In embodiments wherein the genomic DNA of such donor cells is damaged by UV treatment, the methods of making the non-dividing cells generally involves: 1) determining the sensitivity of a strain to UV treatment, and 2) determining the conjugation competency for the UV irradiated cells. The objective is to identify UV treatment conditions that reduce or eliminate the survival (e.g., ability to divide and grow) of the cells, while maintaining the competency of the cells to conjugate. In embodiments wherein the non-dividing cells are to be used for another purpose (e.g., one not involving conjugation), whatever competency is required for that purpose can be the competency measured in step 2.

I. Test of UV Sensitivity

To determine the UV sensitivity of a strain,

1. Transformation with conjugative plasmid. A conjugative plasmid comprising a selectable marker, such as an antibiotic resistance gene (e.g., derivatives of RK2, R6K, F, etc., Lanka, Annu Rev Biochem, 64:141-169 [1995]; Grohmann et al., Microbio Mol Biol Rev, 67:277-301 [2003]) is introduced into each test strain using standard procedures for bacterial transformation. Transformants that contain the selectable marker (e.g., that grow in the presence of the corresponding antibiotic) are selected.

2. Growth of Transformants. The transformants selected in Step 1 (i.e., bacteria carrying one of the conjugative plasmids tested in the step 1) are grown to the stationary phase in a liquid medium. Cells are then spun down and the pellet is washed with a solution such as 0.9% NaCl.

Alternatively, the transformants may be grown on solid media, such as an agar plate. If the agar plate method is used, the resulting colonies are scraped from the plate after growth and are re-suspended in a solution such as 0.9% NaCl.

3. OD Measurement. Measure the OD₆₀₀ of the cells in the solution of the previous step. If the OD₆₀₀ is not 1.0, adjust the OD₆₀₀ to 1.0 using the same solution used to resuspend the cells (e.g., 0.9% NaCl.)

As indicated above, the solution used for washing, cell suspension, and dilution need not be NaCl. For example, the solution may be any solution that is known to be compatible with a particular application, e.g. a phosphate buffer such as might be used for biotherapeutics.

4. UV Irradiation. Transfer an aliquot of the bacterial cell suspension, e.g., 5-10 mls, into a Petri dish, and place the dish on a shaker such as a rotary shaker. Start the rotary shaker at the speed of 60 rpm. The volume of the aliquot may be chosen based on the of the size of the Petri dishes used. Changes in the volume of cell suspension, the size of Petri dish, distance between the UV light source and Petri dish, and rpm of the shaker may alter the observed optimum time of UV irradiation.

Place a UV lamp (e.g., 302 nm wavelength) over the Petri dish and irradiate the cell suspension for different lengths of time (e.g., irradiate different aliquots for times from 0 seconds to up to 5 minutes, in 20-second increments, being sure to keep the other variables [e.g., volume of solution, size of dish, lamp distance, rpm], the same each time). After UV irradiation, place the cell suspensions on ice.

5. Test viability. To determine the survival of the cells at each UV irradiation time point, make a serial dilution of the cell suspension from each time point, and grow on an agar plate to monitor colony-forming units.

Generally, the longer the UV irradiation is applied, the lower the rate of survival becomes. In the event that the shortest time of UV irradiation completely abolished both survival and conjugation competency (measured as described below), one would reduce the dosage of irradiation, e.g., by reducing the intensity of the UV light, increasing the wave length of the UV light, reducing the irradiation time, increasing the distance between Petri dish and UV light, and/or changing the solution used to re-suspend the cells. Conversely, if the longest time of UV irradiation did not sufficiently reduce or eliminate survival, one would increase the dosage of irradiation by increasing the intensity of UV light, reducing the wave length of the UV light, increasing irradiation time, reducing the distance between Petri dish and UV light, or changing the solution used to re-suspend the cells.

II. Test Conjugation Competency

As indicated above, the UV-irradiated cells made in Part I carry one of the conjugative plasmids (from Part 1 protocol, step 1). The conjugation competency of the UV-irradiated cells can be tested using standard laboratory E. coli as a recipient. Conjugation efficiencies of UV treated cells may be compared to the conjugation efficiencies of the same preparation of bacteria that has not been UV-irradiated (e.g., the 0 timepoint). An example procedure for testing conjugation testing is according to the filter conjugation as described in the Description of FIG. 1.

After the tests of Part I and Part II, one would generally select a UV-irradiation time that results in cells having low or no survival when cultured, that have maintained sufficient conjugation competency (or other competency of interest) for the intended purpose. Strains wherein conjugation competency (or other competency of interest) is severely reduced or abolished by UV irradiation conditions that fail to sufficiently reduce survival will generally be less preferred strains for production of conjugation-competent non-dividing cells using the combination of plasmid/bacterium/UV-irradiation condition tested. Conversely, strains that retain a high level of competency under conditions that essentially abolish survival will generally be preferred candidates for production of conjugation-competent non-dividing cells using the combination of plasmid/bacterium/UV-irradiation condition tested. Cells produced under conditions selected according this procedure can be stored as described in Example 5.

The following examples demonstrate that the non-dividing cells of the present invention can be used to kill target cells in and on a variety of plant and animal-derived biological samples. For Examples 8 through 12, conjugation was conducted essentially as described in part B of Example 2, modified as follows. Non-dividing cells carrying the indicated plasmid were mixed with an equal amount of the indicated target bacterial cells, and the mixture was spun down to a pellet. For the solid biological samples, the pellet of mixed cells was re-suspended in a small volume of saline, and spread on the indicated sample surface. For the test in blood plasma, the cells were re-suspended directly in the plasma. Samples were incubated at 37° C. for 1 hour. After the incubation, each mixture of bacteria was eluted with small volume of saline, they were serially diluted, and survival of the target bacteria was monitored by growth them on nutrient-rich agar plates containing appropriate antibiotics. Each of the plasmids described below carries a tetracycline-resistance determinant, while each target strain carries a rifampicin-resistance determinant on the chromosome. The target bacteria that receive the plasmid (called “exconjugants”) can thus be selected on plates containing both tetracycline and rifampicin.

Neither the bacterial cells used to make the non-dividing cells nor the target cells can grow in the presence of both antibiotics. The viability of donor and target cells can be obtained by serial-dilution onto single-antibiotic plates containing only tetracycline or rifampicin, respectively.

### Example 8

### Bacterial Killing by Conjugative Transfer from Non-Dividing Cells on a Flower Surface

A. Rifampicin-Resistant Escherichia coli O157:H7

Target strain: Rifampicin-resistant Escherichia coli O157:H7
Donor strain A:
   E. coli CON4-11c non-dividing (ND) cells carrying a self-transmissible plasmid without colE3 (control group)
   CON4-11c: a derivative of E. coli S17-1, C600 [RK2-2-Tet::Mu Kan::Tn7] lacIq, immE3
   Plasmid (pCON4-45) feature: self-transmissible, selectable markers (AmpR, TetR)
Donor strain B:
   E. coli CON4-11c non-dividing (ND) cells carrying a non-self-transmissible plasmid with colE3:
   CON4-11c: a derivative of E. coli S17-1, C600 [RK2-2-Tet::Mu Kan::Tn7] lacIq, immE3
   Plasmid (pCON4-47) features: self-transmissible, selectable markers (KanR, AmpR, TetR), colE3

Donor strain B contains the antibacterial protein colicin E3 (colE3), which kills the target bacteria upon conjugative transfer of the plasmid.

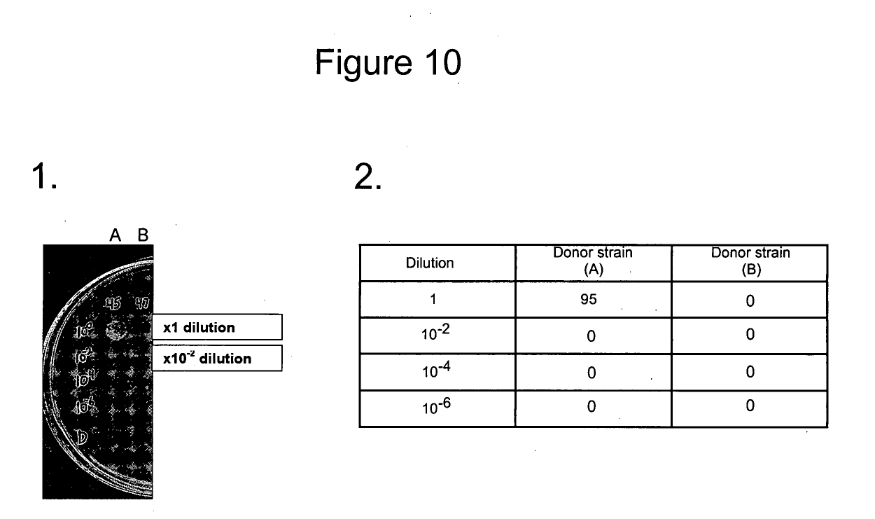

Equal amounts of donor and recipient cells were spotted onto a flower surface for conjugation. After incubation, cells were removed, serially diluted, and spotted on LB-antibiotic plates for measuring colony forming units. The results are shown in FIG. 10. As indicated in the summary in the Table of FIG. 10, no surviving colonies were detected after conjugation and killing using the self-transmissible plasmid.

B. Salmonella enterica Serotype Typhimurium

Target strain: Rifampicin-resistant Salmonella Typhimurium (also known as Salmonella enterica serotype Typhimurium)
Donor strain A:
   E. coli CON4-11c non-dividing (ND) cells carrying a self-transmissible plasmid without colE3 (control group)
   CON4-11c: a derivative of E. coli S17-1, C600 [RK2-2-Tet::Mu Kan::Tn7] lacIq, immE3
   Plasmid (pCON4-45) feature: self-transmissible, selectable markers (AmpR, TetR)
Donor strain B:
   E. coli CON4-11c non-dividing (ND) cells carrying a self-transmissible plasmid with colE3
   CON4-11c: a derivative of E. coli S17-1, C600 [RK2-2-Tet::Mu Kan::Tn7] lacIq, immE3
   Plasmid (pCON4-47) feature: self-transmissible, selectable markers (KanR, AmpR, TetR), colE3

Donor strain B contains the antibacterial protein colicin E3 (colE3), which kills the target bacteria upon conjugative transfer of the plasmid.

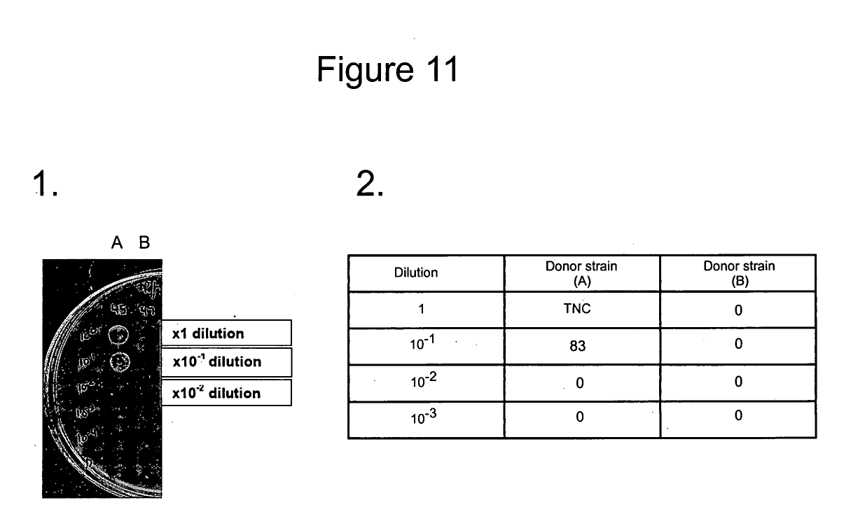

Equal amounts of donor and recipient cells were spotted onto a flower surface for conjugation. After incubation, cells were removed, serially diluted, and spotted on LB-antibiotic plates for measuring colony forming units. The results are shown in FIG. 11. As indicated in the summary in the Table of FIG. 11, no surviving colonies were detected after conjugation and killing using the self-transmissible plasmid.

### Example 9

### Bacterial Killing by Conjugative Transfer from ND Cells on a Leaf Surface

A. Rifampicin-Resistant Escherichia coli O157:H7

Target strain: Rifampicin-resistant Escherichia coli O157:H7
Donor strain A:
   E. coli CON4-11c non-dividing (ND) cells carrying a self-transmissible plasmid without colE3 (control group)
   CON4-11c: a derivative of E. coli S17-1, C600 [RK2-2-Tet::Mu Kan::Tn7] lacIq, immE3
   Plasmid (pCON4-45) feature: self-transmissible, selectable markers (AmpR, TetR)
Donor strain B:
   E. coli CON4-11c non-dividing (ND) cells carrying a non-self-transmissible plasmid with colE3
   CON4-11c: a derivative of E. coli S17-1, C600 [RK2-2-Tet::Mu Kan::Tn7] lacIq, immE3
   Plasmid (pCON4-47) feature: self-transmissible, selectable markers (KanR, AmpR, TetR), colE3

Donor strain B contains the antibacterial protein colicin E3 (colE3), which kills the target bacteria upon conjugative transfer of the plasmid.

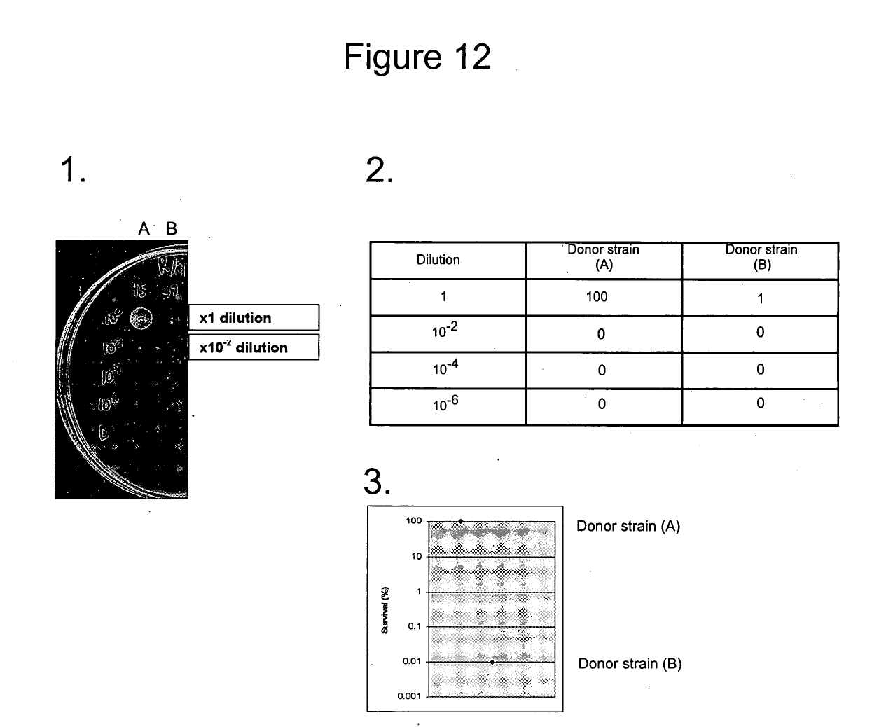

Equal amounts of donor and recipient cells were spotted onto a leaf surface for conjugation. After incubation, cells were removed, serially diluted, and spotted on LB-antibiotic plates for measuring colony forming units. The results are shown in FIG. 12. As indicated in the summary in the Table of FIG. 12, no surviving colonies were detected after conjugation and killing using the self-transmissible plasmid. The results are shown in FIG. 10.

B. Salmonella enterica Serotype Typhimurium

Target strain: Rifampicin-resistant Salmonella Typhimurium (also known as Salmonella enterica serotype Typhimurium)
Donor strain A:
   E. coli CON4-11c non-dividing (ND) cells carrying a self-transmissible plasmid without colE3 (control group)
   CON4-11c: a derivative of E. coli S17-1, C600 [RK2-2-Tet::Mu Kan::Tn7] lacIq, immE3
   Plasmid (pCON4-45) feature: self-transmissible, selectable markers (AmpR, TetR)
Donor strain B:
   E. coli CON4-11c non-dividing (ND) cells carrying a self-transmissible plasmid with colE3
   CON4-11c: a derivative of E. coli S17-1, C600 [RK2-2-Tet::Mu Kan::Tn7] lacIq, immE3
   Plasmid (pCON4-47) feature: self-transmissible, selectable markers (KanR, AmpR, TetR), colE3

Donor strain B contains the antibacterial protein colicin E3 (colE3), which kills the target bacteria upon conjugative transfer of the plasmid.

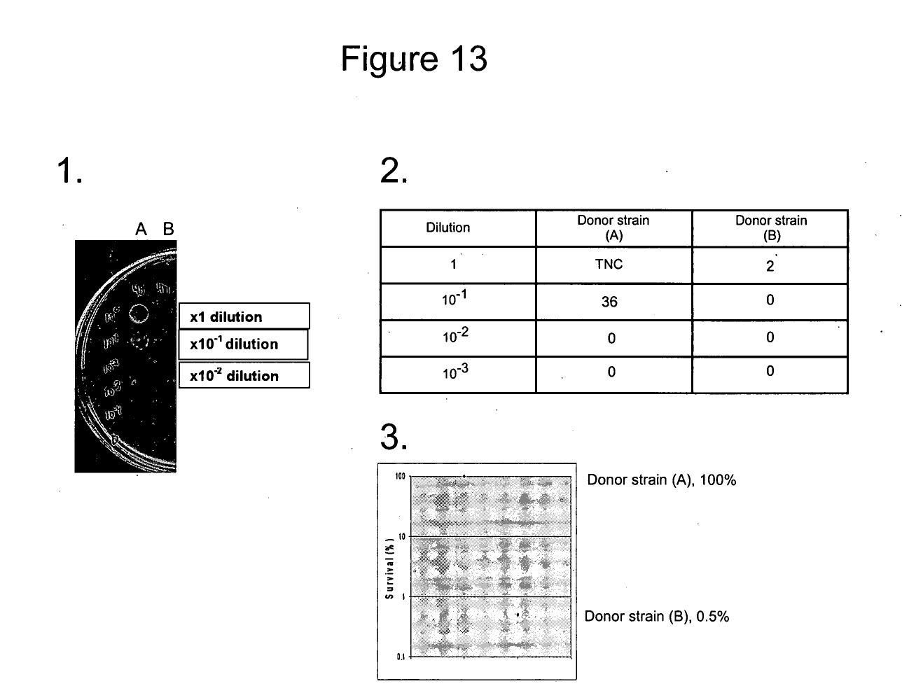

Equal amounts of donor and recipient cells were spotted onto a leaf surface for conjugation. After incubation, cells were removed, serially diluted, and spotted on LB-antibiotic plates for measuring colony forming units. The results are shown in FIG. 13. As indicated in the graph in panel 3 showing the comparison of the survival rates, 100% of the target bacteria survived conjugation with the control plasmid (A), while only 0.5% of the target bacteria survived when the self-transmissible plasmid (B) was used.

### Example 10

### Bacterial Killing by Conjugative Transfer from ND Cells on a Potato Surface

A. Rifampicin-Resistant Escherichia coli O157:H7

Target strain: Rifampicin-resistant Escherichia coli O157:H7
Donor strain A:
   E. coli CON4-11c non-dividing (ND) cells carrying a self-transmissible plasmid without colE3 (control group)
   CON4-11c: a derivative of E. coli S17-1, C600 [RK2-2-Tet::Mu Kan::Tn7] lacIq, immE3
   Plasmid (pCON4-45) feature: self-transmissible, selectable markers (AmpR, TetR)
Donor strain B:
   E. coli CON4-11c non-dividing (ND) cells carrying a non-self-transmissible plasmid with colE3
   CON4-11c: a derivative of E. coli S17-1, C600 [RK2-2-Tet::Mu Kan::Tn7] lacIq, immE3
   Plasmid (pCON4-47) feature: self-transmissible, selectable markers (KanR, AmpR, TetR), colE3

Donor strain B contains the antibacterial protein colicin E3 (colE3), which kills the target bacteria upon conjugative transfer of the plasmid.

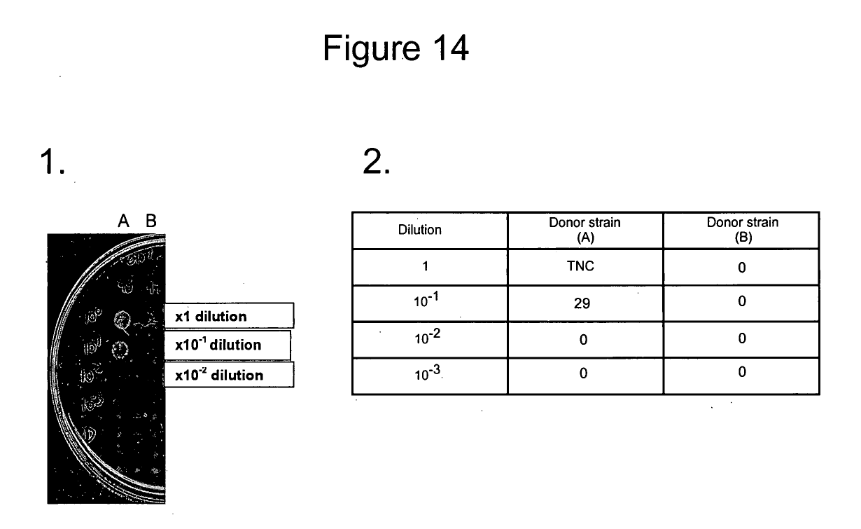

Equal amounts of donor and recipient cells were spotted onto a potato surface for conjugation. After incubation, cells were removed, serially diluted, and spotted on LB-antibiotic plates for measuring colony forming units. The results are shown in FIG. 14. As indicated in the summary in the Table of FIG. 14, no surviving colonies were detected after conjugation and killing using the self-transmissible plasmid.

B. Salmonella enterica Serotype Typhimurium

Target strain: Rifampicin-resistant Salmonella Typhimurium (also known as Salmonella enterica serotype Typhimurium)
Target strain: Rifampicin-resistant Salmonella Typhimurium (also known as Salmonella enterica serotype Typhimurium)
Donor strain A:
   E. coli CON4-11c non-dividing (ND) cells carrying a self-transmissible plasmid without colE3 (control group)
   CON4-11c: a derivative of E. coli S17-1, C600 [RK2-2-Tet::Mu Kan::Tn7] lacIq, immE3
   Plasmid (pCON4-45) feature: self-transmissible, selectable markers (AmpR, TetR)
Donor strain B:
   E. coli CON4-11c non-dividing (ND) cells carrying a non-self-transmissible plasmid with colE3
   CON4-11c: a derivative of E. coli S17-1, C600 [RK2-2-Tet::Mu Kan::Tn7] lacIq, immE3
   Plasmid (pCON4-44) feature: non-self-transmissible, selectable markers (KanR, TetR), colE3
Donor strain C:
   E. coli CON4-11c non-dividing (ND) cells carrying a self-transmissible plasmid with colE3
   CON4-11c: a derivative of E. coli S17-1, C600 [RK2-2-Tet::Mu Kan::Tn7] lacIq, immE3
   Plasmid (pCON4-47) feature: self-transmissible, selectable markers (KanR, AmpR, TetR), colE

Donor strains B and C contain the antibacterial protein colicin E3 (colE3), which kills the target bacteria upon conjugative transfer of the plasmid.

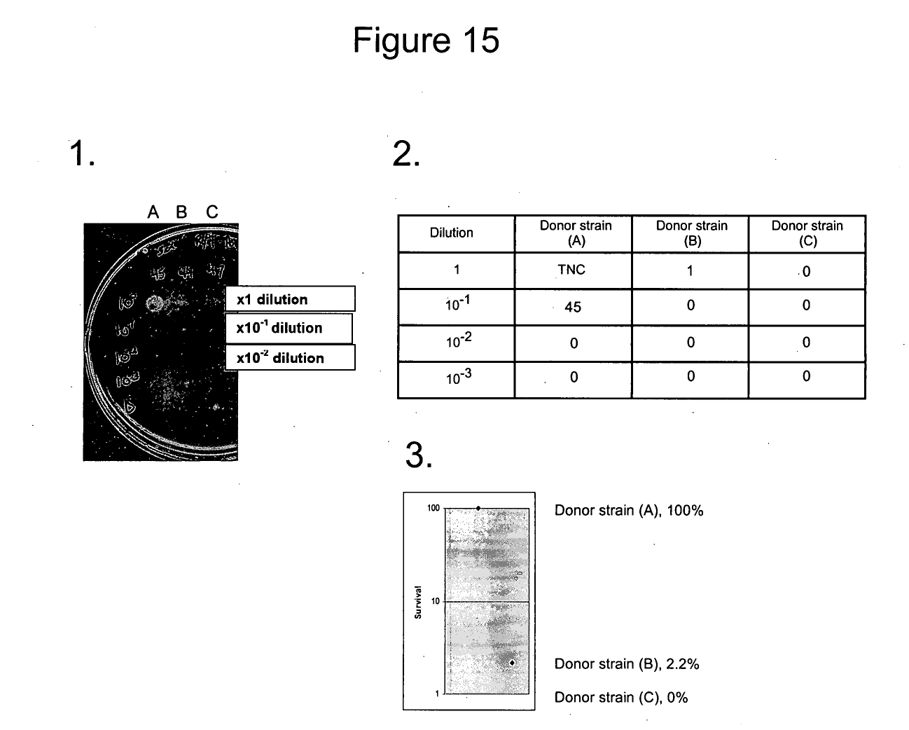

Equal amounts of donor and recipient cells were spotted onto a leaf surface for conjugation. After incubation, cells were removed, serially diluted, and spotted on LB-antibiotic plates for measuring colony forming units. The results are shown in FIG. 15. As indicated in the graph in panel 3 showing the comparison of the survival rates, 100% of the target bacteria survived conjugation with the control plasmid (A) and only 2.2% survived conjugation with the non self-transmissible plasmid (B). None of the target bacteria survived when the self-transmissible plasmid (C) was used.

### Example 11

### Bacterial Killing by Conjugative Transfer from ND Cells on the Surface of Meat

A. Rifampicin-Resistant Escherichia coli O157:H7

Target strain: Rifampicin-resistant Escherichia coli O157:H7
Donor strain A:
   E. coli CON4-11c non-dividing (ND) cells carrying a self-transmissible plasmid without colE3 (control group)
   CON4-11c: a derivative of E. coli S17-1, C600 [RK2-2-Tet::Mu Kan::Tn7] lacIq, immE3
   Plasmid (pCON4-45) feature: self-transmissible, selectable markers (AmpR, TetR)
Donor strain B:
   E. coli CON4-11c non-dividing (ND) cells carrying a non-self-transmissible plasmid with colE3
   CON4-11c: a derivative of E. coli S17-1, C600 [RK2-2-Tet::Mu Kan::Tn7] lacIq, immE3
   Plasmid (pCON4-47) feature: self-transmissible, selectable markers (KanR, AmpR, TetR), colE3

Donor strain B contains the antibacterial protein colicin E3 (colE3), which kills the target bacteria upon conjugative transfer of the plasmid.

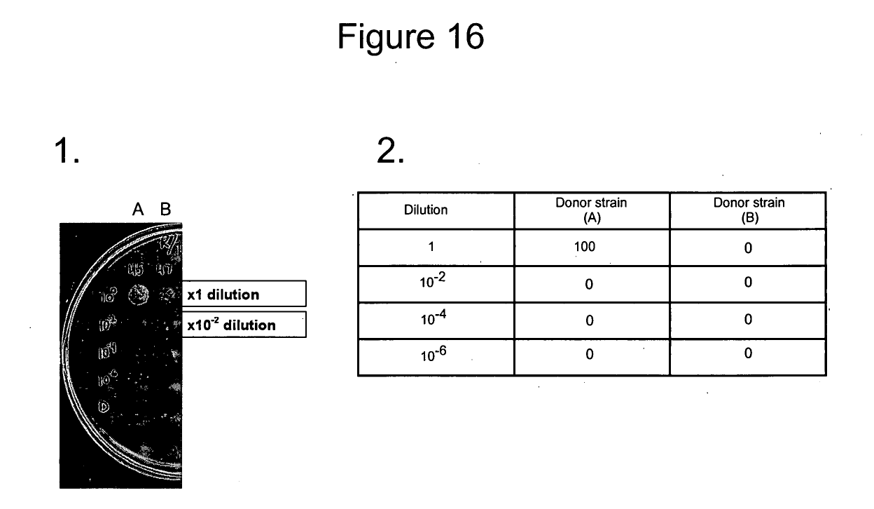

Equal amounts of donor and target cells were spotted onto a meat surface for conjugation. After incubation, cells were removed, serially diluted, and spotted on LB-antibiotic plates for measuring colony forming units. The results are shown in FIG. 16. As indicated in the Table showing the comparison of the survival rates, 100% of the target bacteria survived conjugation with the control plasmid (A), while none of the target bacteria survived when the self-transmissible plasmid (B) was used.

B. Salmonella enterica Serotype Typhimurium

Target strain: Rifampicin-resistant Salmonella Typhimurium (also known as Salmonella enterica serotype Typhimurium)
Donor strain A:
   E. coli CON4-11c non-dividing (ND) cells carrying a self-transmissible plasmid without colE3 (control group)
   CON4-11c: a derivative of E. coli S17-1, C600 [RK2-2-Tet::Mu Kan::Tn7] lacIq, immE3
   Plasmid (pCON4-45) feature: self-transmissible, selectable markers (AmpR, TetR)
Donor strain B:
   E. coli CON4-11c non-dividing (ND) cells carrying a self-transmissible plasmid with colE3
   CON4-11c: a derivative of E. coli S17-1, C600 [RK2-2-Tet::Mu Kan::Tn7] lacIq, immE3
   Plasmid (pCON4-47) feature: self-transmissible, selectable markers (KanR, AmpR, TetR), colE3

Donor strain B contains the antibacterial protein colicin E3 (colE3), which kills the target bacteria upon conjugative transfer of the plasmid.

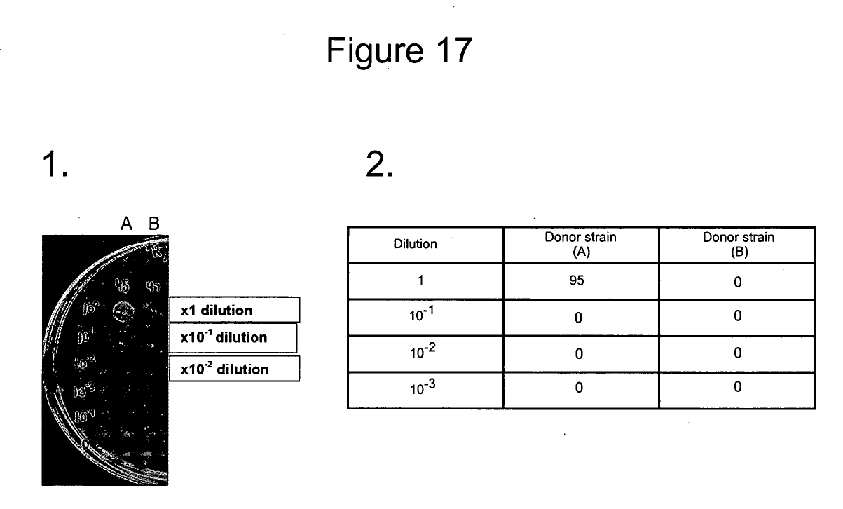

Equal amounts of donor and target cells were spotted onto a meat surface for conjugation. After incubation, cells were removed, serially diluted, and spotted on LB-antibiotic plates for measuring colony forming units. The results are shown in FIG. 17. As indicated in the Table showing the comparison of the survival rates, 100% of the target bacteria survived conjugation with the control plasmid (A), while none of the target bacteria survived when the self-transmissible plasmid (B) was used.

### Example 12

### Bacterial Killing by Conjugative Transfer from ND Cells in Blood Plasma

A. Rifampicin-Resistant Escherichia coli O157:H7

Target strain: Rifampicin-resistant Escherichia coli O157:H7
Donor strain A:
   E. coli CON4-11c non-dividing (ND) cells carrying a self-transmissible plasmid without colE3 (control group)
   CON4-11c: a derivative of E. coli S17-1, C600 [RK2-2-Tet::Mu Kan::Tn7] lacIq, immE3
   Plasmid (pCON4-45) feature: self-transmissible, selectable markers (AmpR, TetR)
Donor strain B:
   E. coli CON4-11c non-dividing (ND) cells carrying a non-self-transmissible plasmid with colE3
   CON4-11c: a derivative of E. coli S17-1, C600 [RK2-2-Tet::Mu Kan::Tn7] lacIq, immE3
   Plasmid (pCON4-47) feature: self-transmissible, selectable markers (KanR, AmpR, TetR), colE3

Donor strain B contains the antibacterial protein colicin E3 (colE3), which kills the target bacteria upon conjugative transfer of the plasmid.

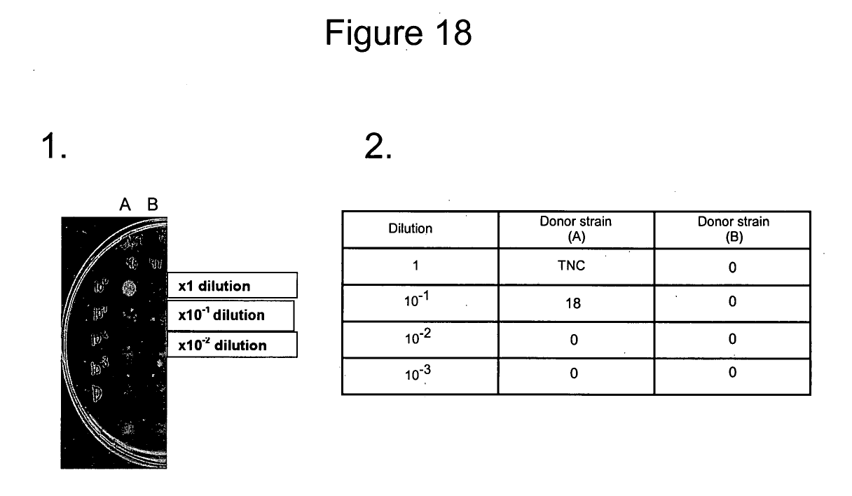

Equal amounts of donor and recipient cells were diluted into blood plasma for conjugation. After incubation, cells were removed, serially diluted, and spotted on LB-antibiotic plates for measuring colony forming units. The results are shown in FIG. 18. As indicated in the Table showing the comparison of the survival rates, 100% of the target bacteria survived conjugation with the control plasmid (A), while none of the target bacteria survived when the self-transmissible plasmid (B) was used.

B. Salmonella enterica Serotype Typhimurium

Target strain: Rifampicin-resistant Salmonella Typhimurium (also known as Salmonella enterica serotype Typhimurium)
Donor strain A:
   E. coli CON4-11c non-dividing (ND) cells carrying a self-transmissible plasmid without colE3 (control group)
   CON4-11c: a derivative of E. coli S17-1, C600 [RK2-2-Tet::Mu Kan::Tn7] lacIq, immE3
   Plasmid (pCON4-45) feature: self-transmissible, selectable markers (AmpR, TetR)
Donor strain B:
   E. coli CON4-11c non-dividing (ND) cells carrying a non-self-transmissible plasmid with colE3
   CON4-11c: a derivative of E. coli S17-1, C600 [RK2-2-Tet::Mu Kan::Tn7] lacIq, immE3
   Plasmid (pCON4-44) feature: non-self-transmissible, selectable markers (KanR, TetR), colE3
Donor strain C:
   E. coli CON4-11c non-dividing (ND) cells carrying a self-transmissible plasmid with colE3
   CON4-11c: a derivative of E. coli S17-1, C600 [RK2-2-Tet::Mu Kan::Tn7] lacIq, immE3
   Plasmid (pCON4-47) feature: self-transmissible, selectable markers (KanR, AmpR, TetR), colE3

Donor strains B and C contain the antibacterial protein colicin E3 (colE3), which kills the target bacteria upon conjugative transfer of the plasmid.

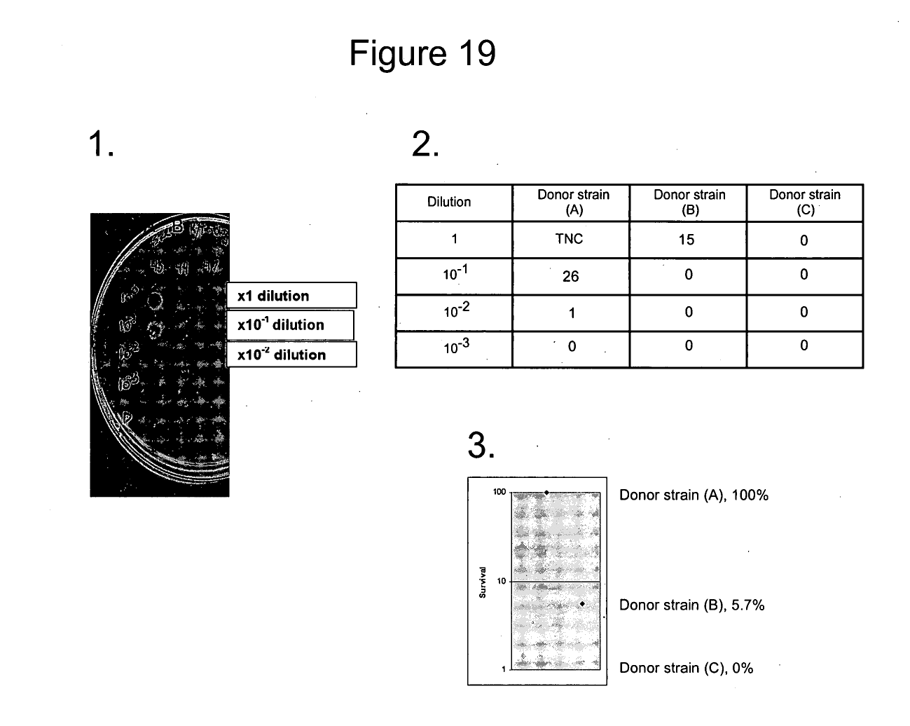

Equal amounts of donor and recipient cells were diluted into blood plasma for conjugation. After incubation, cells were removed, serially diluted, and spotted on LB-antibiotic plates for measuring colony forming units. The results are shown in FIG. 19. As indicated in the graph in panel 3 showing the comparison of the survival rates, 100% of the target bacteria survived conjugation with the control plasmid (A) and only 5.7% survived conjugation with the non self-transmissible plasmid (B). None of the target bacteria survived when the self-transmissible plasmid (C) was used.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A method of producing a conjugation-competent non-dividing cell comprising:
a) providing a conjugation-competent bacterial cell deficient in one or more DNA repair systems such that the cell's ability to repair DNA damage is substantially impaired;
b) exposing said bacterial cell to DNA damaging conditions whereby the chromosomal DNA of said bacterial cell is damaged sufficiently to prevent cell division; and
c) treating said bacterial cell having damaged chromosomal DNA under conditions wherein conjugation competency is preserved.

2. The method of claim 1, wherein said DNA damaging conditions comprise irradiation.

3. The method of claim 2 wherein said irradiation comprises irradiation by ultraviolet light.

4. The method of claim 2, wherein said irradiation comprises irradiation by gamma rays.

5. The method of claim 1, wherein said treating comprises chilling.

6. The method of claim 5, wherein said chilling comprises chilling to a temperature of about 0° C. to 10° C.

7. The method of claim 6, wherein said chilling comprises chilling to a temperature of about 0° C. to 5° C.

8. The method of claim 7, wherein said chilling comprises chilling to a temperature of about 0° C. to 1° C.

9. The method of claim 5, wherein said chilling comprising freezing to a temperature below 0° C.

10. The method of claim 2, wherein said treating comprises chilling immediately after said irradiation.

11. The method of claim 1, wherein said bacterial cell comprises a mutation in said one or more DNA repair systems, wherein said mutation impairs the function of said one or more DNA repair systems.

12. The method of claim 11, wherein said mutation is in a gene selected from the group consisting of recA, uvrA and phrB.

13. The method of claim 1, wherein said bacterial cell is Gram-positive.

14. The method of claim 1, wherein said bacterial cell is Gram-negative.

15. The method of claim 1, where said bacterial cell is selected from the group consisting of Lactobacillis acidophilis, Lactococcus lactis, Lactobacillus plantarum, Bacillus subtilis, Staphylococcus species, Streptococcus species.

16. The method of claim 1, where said bacterial cell is selected from the group consisting of Escherichia coli, Helicobacter pylori, Pseudomonas aeruginosa, Haemophilus influenzae, somnus and ducreyi, Klebsiella pneumoniae

17. The method of claim 1, wherein said non-dividing cell has conjugation-competency that is substantially similar to the conjugation competency of said bacterial cell.

18. The method of claim 1, wherein said bacterial cell comprises tra genes encoding components for conjugation.

19. The method of claim 18, wherein said bacterial cell further comprises a transmissible element.

20. The method of claim 18, wherein said transmissible element is DNA.

21. The method of claim 20, wherein said DNA is a plasmid.

22. The method of claim 20, wherein said DNA comprises an origin of transfer.

23. The method of claim 22, wherein said origin of transfer is from a Gram-negative bacterium.

24. The method of claim 22, wherein said origin of transfer is from a Gram-positive bacterium.

25. The method of claim 18, wherein said one or more tra genes are located on the chromosomal DNA of said bacterial cell.

26. The method of claim 21, wherein one or more tra genes are located on said plasmid.

27. The method of claim 21, wherein said one or more tra genes are located on said plasmid and on said chromosomal DNA.

28. A method of producing a conjugation-competent non-dividing cells comprising:
a) providing conjugation-competent bacterial cells deficient in one or more DNA repair systems such that the cell's ability to repair DNA damage is substantially impaired;
b) irradiating said conjugation-competent bacterial cells, whereby the chromosomal DNA of said bacterial cell is damaged sufficiently to prevent cell division, wherein said irradiation comprises passing said bacterial cells past a radiation source to provide a controlled dosage of radiation to said bacterial cells; and
c) treating said bacterial cells having damaged chromosomal DNA under conditions wherein conjugation competency is preserved.

29. The method of claim 28, wherein the dosage of irradiation received by said bacterial cells is controlled by the rate at which said bacterial cells and said radiation source pass each other.

30. The method of claim 29, wherein said radiation source is stationary and said bacterial cells are moved past said radiation source.

31. A composition comprising a non-dividing cell, wherein said non-dividing cell is a bacterial cell deficient in one or more DNA repair systems, wherein the bacterial cell has been exposed to DNA damaging conditions wherein the chromosomal DNA of said bacterial cell is damaged sufficiently to prevent cell division, and wherein said bacterial cell has further been treated under conditions wherein conjugation competency is preserved.

32. The composition of claim 31, wherein said non-dividing cell further comprises:
a. one or more tra genes conferring upon the non-dividing cell the ability to conjugatively transfer a transmissible plasmid to at least one recipient bacterial cell;
b. at least one transmissible plasmid, wherein said transmissible plasmid comprises an origin of transfer from which conjugative transfer of the transmissible plasmid initiates from the non-dividing cell to said at least one recipient cell.

33. The composition of claim 32, wherein said transmissible plasmid further comprises a gene encoding a toxin.

34. The composition of claim 33, wherein said gene encoding a toxin encodes a bacterial colicin.

35. The composition of claim 34, wherein said colicin is selected from the group consisting of Colicin D, Colicin E3, and Colicin E7.

36. The composition of claim 32, wherein said one or more tra genes are on the chromosomal DNA of said non-dividing cell.

37. The composition of claim 32 wherein said one or more tra genes are on said at least one transmissible plasmid.

38. The composition of claim 32, wherein said chromosomal DNA of said non-dividing cell and said transmissible plasmid each comprise one or more of said one or more tra genes.

39. The composition of claim 32, wherein said non-dividing cell further comprises a helper plasmid, wherein one or more of said one or more tra genes are on said helper plasmid.

40. The composition of claim 32 further comprising a preservative.

41. The composition of claim 40, wherein said preservative is a cryopreservative.

42. The composition of claim 40, wherein said preservative is selected from the group consisting of trehalose, glycerol, sucrose, DMSO and ethylene glycol.

43. The composition of claim 32, wherein said composition is freeze-dried.
